SEQUENCE LISTING (53 sequences)

SEQ ID NO: 1 - DNA, 375 bp, synthetic construct
  misc_feature 1..375: note = 3B9 VL1-VH nucleic acid sequence
  source 1..375: mol_type = other DNA
caggtgcagctggtgcagtctggggctgaggtgaagaagcctgggtcctcggtgaaggtctcctgcaaggcttctggaggcaccttcagcagctatgctatcagctgggtgcgccaggcccctggacaagggcttgagtggatgggagggatcatccctctctttgatacaacaaagtacgcacagcagttccagggcagagtcacgattaccgcggacgaatccacgagcacagcctacatggagctgagcagcctgagatctgaggacacggccgtattttactgtgcgagagatcgggatattttgactgattattatcccatgggcggtatggacgtctggggccaagggaccacggtcaccgtctcctca

SEQ ID NO: 2 - protein, 125 aa, synthetic construct
  REGION 1..125: note = 3B9 VL1-VH amino acid sequence
QVQLVQSGAEVKKPGSSVKVSCKASGGTFSSYAISWVRQAPGQGLEWMGGIIPLFDTTKYAQQFQGRVTITADESTSTAYMELSSLRSEDTAVFYCARDRDILTDYYPMGGMDVWGQGTTVTVSS

SEQ ID NO: 3 - DNA, 324 bp, synthetic construct
  misc_feature 1..324: note = 39B9 VL1-VL nucleic acid sequence
  source 1..324: mol_type = other DNA
gccatccagttgacccagtctccatcctccctgtctgcatctgtaggagacagagtcaccatcacttgccgggcaagtcagggcattagcagtgttttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtctaatagttacccgctcactttcggcggagggaccaaggtggagatcaaacgt

SEQ ID NO: 4 - protein, 108 aa, synthetic construct
  REGION 1..108: note = 39B9 VL1-VL amino acid sequence
AIQLTQSPSSLSASVGDRVTITCRASQGISSVLAWYQQKPGKAPKLLIYDASSLESGVPSRFSGSGSGTDFTLTISSLQPEDFATYYCQQSNSYPLTFGGGTKVEIKR

SEQ ID NO: 5 - DNA, 324 bp, synthetic construct
  misc_feature 1..324: note = 39B9 VL5-VL nucleic acid sequence
  source 1..324: mol_type = other DNA
gacatcctgatgacccagtctccatcctccctgtctgcatctgtaggagacagagtcaccatcacttgtcgggcgagtcaggatattagcagctggttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtctaatagttacccgctcactttcggcggagggaccaaggtggagatcaaacga

SEQ ID NO: 6 - protein, 108 aa, synthetic construct
  REGION 1..108: note = 39B9 VL5-VL amino acid sequence
DILMTQSPSSLSASVGDRVTITCRASQDISSWLAWYQQKPGKAPKLLIYDASSLESGVPSRFSGSGSGTDFTLTISSLQPEDFATYYCQQSNSYPLTFGGGTKVEIKR

SEQ ID NO: 7 - DNA, 357 bp, synthetic construct
  misc_feature 1..357: note = 12A VH nucleic acid sequence
  source 1..357: mol_type = other DNA
caggtgcagctggtggagtcttggggaggcgtggtccagcctgggaggtccctgagactctcctgtgcagcgtctggattcaccttcagtaactatgacatgtactgggtccgccaggctccaggcaaggggctggagtgggtggcagttatattagatgatggaaataataattactacgcagactccgtgaagggccgattcaccatctccagagacaattccaagaaaaaggtgtatctgcaaatgaatagcctgagagctgaggacacggctgtgtattactgtgtgagagcgtcccctaactggggtcttcttgacttctggggccagggaaccctggtcaccgtctcgagt

SEQ ID NO: 8 - protein, 119 aa, synthetic construct
  REGION 1..119: note = 12A VH amino acid sequence
QVQLVESWGGVVQPGRSLRLSCAASGFTFSNYDMYWVRQAPGKGLEWVAVILDDGNNNYYADSVKGRFTISRDNSKKKVYLQMNSLRAEDTAVYYCVRASPNWGLLDFWGQGTLVTVSS

SEQ ID NO: 9 - DNA, 324 bp, synthetic construct
  misc_feature 1..324: note = 12A VL nucleic acid sequence
  source 1..324: mol_type = other DNA
gaaattgtgttgacacagtctccatcctcactgtctgcatctgtaggagacagagtcaccatcacttgtcgggcgagtcagggtattagcagctggttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccgatcaccttcggccaagggacacgactggagattaaacgt

SEQ ID NO: 10 - protein, 108 aa, synthetic construct
  REGION 1..108: note = 12A VL amino acid sequence
EIVLTQSPSSLSASVGDRVTITCRASQGISSWLAWYQQKPGKAPKLLIYDASSLESGVPSRFSGSGSGTDFTLTISSLQPEDFATYYCQQFNSYPITFGQGTRLEIKR

SEQ ID NO: 11 - DNA, 357 bp, synthetic construct
  misc_feature 1..357: note = 5C VH nucleic acid sequence
  source 1..357: mol_type = other DNA
caggtgcagctggtgcagtctgggggaggcgtggtccagcctgggaggtccctgagactctcctgtgcagcctctggattcatcttcagtagctatgacatgtactgggtccgccaggctccaggcaaggggctggagtgggtggcagttatattatatgatggaaataataaatactacgcagactccgtgaagggccgattcaccatctccagagacaattccaagaacacggtgtatctgcaaatgaacagcctgagagctgaggacacggctgtgtattactgtgtgagagcgtcccctaactggggtctttttgacttctggggccagggaaccctggtcaccgtctcgagt

SEQ ID NO: 12 - protein, 119 aa, synthetic construct
  REGION 1..119: note = 5C VH amino acid sequence
QVQLVQSGGGVVQPGRSLRLSCAASGFIFSSYDMYWVRQAPGKGLEWVAVILYDGNNKYYADSVKGRFTISRDNSKNTVYLQMNSLRAEDTAVYYCVRASPNWGLFDFWGQGTLVTVSS

SEQ ID NO: 13 - DNA, 324 bp, synthetic construct
  misc_feature 1..324: note = 5C VL nucleic acid sequence
  source 1..324: mol_type = other DNA
gacatccagatgacccagtctccatcctccctgtctgcatctgtaggagacagagtcaccatcacttgccgggcaagtcagggcattagcagtgatttagcctggtatcagcagaaaccagggaaagctcctaagctcctgatgtatgatgcctccagtttggaaagtggggtcccatcaaggttcagcggcagtggatctgggacagatttcactctcaccatcagcagcctgcagcctgaagattttgcaacttattactgtcaacagtttaatagttacccgatcaccttcggccaagggacacgactggagattaaacgt

SEQ ID NO: 14 - protein, 108 aa, synthetic construct
  REGION 1..108: note = 5C VL amino acid sequence
DIQMTQSPSSLSASVGDRVTITCRASQGISSDLAWYQQKPGKAPKLLMYDASSLESGVPSRFSGSGSGTDFTLTISSLQPEDFATYYCQQFNSYPITFGQGTRLEIKR

SEQ ID NO: 15 - protein, 5 aa, synthetic construct
  REGION 1..5: note = 39B9 VH CDR1
SYAIS

SEQ ID NO: 16 - protein, 17 aa, synthetic construct
  REGION 1..17: note = 39B9 VH CDR2
GIIPLFDTTKYAQQFQG

SEQ ID NO: 17 - protein, 19 aa, synthetic construct
  REGION 1..19: note = 39B9 VH CDR3
CARDRDILTDYYPMGGMDV

SEQ ID NO: 18 - protein, 5 aa, synthetic construct
  REGION 1..5: note = 12A VH CDR1
NYDMY

SEQ ID NO: 19 - protein, 17 aa, synthetic construct
  REGION 1..17: note = 12A VH CDR2
VILDDGNNNYYADSVKG

SEQ ID NO: 20 - protein, 13 aa, synthetic construct
  REGION 1..13: note = 12A VH CDR3
CVRASPNWGLLDF

SEQ ID NO: 21 - protein, 5 aa, synthetic construct
  REGION 1..5: note = 5C VH CDR1
SYDMY

SEQ ID NO: 22 - protein, 17 aa, synthetic construct
  REGION 1..17: note = 5C VH CDR2
VILYDGNNKYYADSVKG

SEQ ID NO: 23 - protein, 13 aa, synthetic construct
  REGION 1..13: note = 5C VH CDR3
CVRASPNWGLFDF

SEQ ID NO: 24 - protein, 11 aa, synthetic construct
  REGION 1..11: note = 39B9 VL1 VL CDR1
RASQGISSVLA

SEQ ID NO: 25 - protein, 7 aa, synthetic construct
  REGION 1..7: note = IL-6 antibody VL CDR2
DASSLES

SEQ ID NO: 26 - protein, 9 aa, synthetic construct
  REGION 1..9: note = 39B9 VL1 VL CDR3
QQSNSYPLT

SEQ ID NO: 27 - protein, 11 aa, synthetic construct
  REGION 1..11: note = 39B9 VL5 VL CDR1
RASQDISSWLA

SEQ ID NO: 28 - protein, 11 aa, synthetic construct
  REGION 1..11: note = 12A VL CDR1
RASQGISSWLA

SEQ ID NO: 29 - protein, 9 aa, synthetic construct
  REGION 1..9: note = 12A VL CDR3
QQSNSYPIT

SEQ ID NO: 30 - protein, 11 aa, synthetic construct
  REGION 1..11: note = 5C VL CDR1
RASQGISSVDA

SEQ ID NO: 31 - protein, 9 aa, synthetic construct
  REGION 1..9: note = VL CDR3 motif
  VARIANT 4: note = X is N or Q
QQSXSYPLT

SEQ ID NO: 32 - protein, 17 aa, synthetic construct
  REGION 1..17: note = VH CDR2 motif
  VARIANT 5: note = X is L or A
  VARIANT 7: note = X is D or E
  VARIANT 14: note = X is Q or K
GIIPXFXTTKYAQXFQG

SEQ ID NO: 33 - protein, 16 aa, synthetic construct
  REGION 1..16: note = VH CDR3 motif
  VARIANT 11: note = X is M or L
DRDILTDYYPXGGMDV

SEQ ID NO: 34 - protein, 8 aa, synthetic construct
  REGION 1..8: note = FRW3 mofitf
  VARIANT 4: note = X is F or Y
TAVXYCAR

SEQ ID NO: 35 - protein, 16 aa, synthetic construct
  REGION 1..16: note = NI-1201-WT VH CDR3
DRDILTDYYPMGGMDV

SEQ ID NO: 36 - protein, 8 aa, synthetic construct
  REGION 1..8: note = NI-1201-WT FRW3
TAVFYCAR

SEQ ID NO: 37 - protein, 17 aa, synthetic construct
  REGION 1..17: note = NI-1201-A VL CDR3
GIIPLFDTTKYAQKFQG

SEQ ID NO: 38 - protein, 8 aa, synthetic construct [sequence omitted]

SEQ ID NO: 39 - protein, 16 aa, synthetic construct
  REGION 1..16: note = NI-1201-B VH CDR3
DRDILTDYYPLGGMDV

SEQ ID NO: 40 - protein, 17 aa, synthetic construct
  REGION 1..17: note = NI-1201-C VH CDR2
GIIPAFETTKYAQKFQG

SEQ ID NO: 41 - protein, 9 aa, synthetic construct
  REGION 1..9: note = NI-1201-D VL CDR3
QQSQSYPLT

SEQ ID NO: 42 - protein, 5 aa, synthetic construct
  REGION 1..5: note = anti-CD3 CDRH1
GYGMH

SEQ ID NO: 43 - protein, 17 aa, synthetic construct
  REGION 1..17: note = anti-CD3 CDRH2
VIWYDGSKKYYVDSVKG

SEQ ID NO: 44 - protein, 9 aa, synthetic construct
  REGION 1..9: note = anti-CD3 CDRH3
QMGYWHFDL

SEQ ID NO: 45 - protein, 11 aa, synthetic construct
  REGION 1..11: note = anti-CD3 CDRL1
RASQSVSSYLA

SEQ ID NO: 46 - protein, 7 aa, synthetic construct
  REGION 1..7: note = anti-CD3 CDRL2
DASNRAT

SEQ ID NO: 47 - protein, 10 aa, synthetic construct
  REGION 1..10: note = anti-CD3 CDRL3
QQRSNWPPLT

SEQ ID NO: 48 - protein, 118 aa, synthetic construct [sequence omitted]

SEQ ID NO: 49 - protein, 108 aa, synthetic construct [sequence omitted]

SEQ ID NO: 50 - protein, 448 aa, synthetic construct
  REGION 1..448: note = anti-CD3 HC
QVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 215 aa, synthetic construct
  REGION 1..215: note = anti-CD3 LC
EIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 454 aa, synthetic construct
  REGION 1..454: note = TZLS-501 heavy chain
QVQLVQSGAEVKKPGSSVKVSCKASGGTFSSYAISWVRQAPGQGLEWMGGIIPLFDTTKYAQKFQGRVTITADESTSTAYMELSSLRSEDTAVYYCARDRDILTDYYPMGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 214 aa, synthetic construct
  REGION 1..214: note = TZLS-501 light chain
AIQLTQSPSSLSASVGDRVTITCRASQGISSVLAWYQQKPGKAPKLLIYDASSLESGVPSRFSGSGSGTDFTLTISSLQPEDFATYYCQQSNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0125471 A1
(43) Pub. Date: May 7, 2026

(54) SUBCUTANEOUS ADMINISTRATION OF ANTIBODIES FOR THE TREATMENT OF DISEASE

(71) Applicant: Tiziana Life Sciences PLC, London (GB)

(72) Inventors: Kunwar SHAILUBHAI, Line Lexington, PA (US); Vaseem A. PALEJWALA, Scotch Plains, NJ (US); Jules S. JACOB, Perkasie, PA (US)

(21) Appl. No.: 19/316,286

(22) Filed: Sep. 2, 2025

Related U.S. Application Data
(62) Division of application No. 17/723,098, filed on Apr. 18, 2022, now abandoned.
(60) Provisional application No. 63/175,674, filed on Apr. 16, 2021.

(51) Int. Cl. C07K 16/28; A61K 39/00; A61P 1/16; A61P 3/10; A61P 29/00; C07K 16/24

(52) U.S. Cl. CPC C07K 16/2809; A61P 1/16; A61P 3/10; A61P 29/00; C07K 16/248; A61K 2039/505; C07K 2317/24; C07K 2317/565; C07K 2317/92

(57) ABSTRACT

This invention provides methods of subcutaneous administration of anti-CD3 monoclonal antibodies (mAbs) either alone or in combination with monoclonal antibodies, that recognize the Interleukin-6 (IL-6) and IL-6 receptor (IL-6R) complex for the treatment, prevention or alleviating a symptom of a disease.

### CROSS REFERENCE TO RELATED APPLICATIONS

This application is a divisional application of U.S. patent application Ser. No. 17/723,098, filed Apr. 18, 2022, which claims priority to and the benefit of U.S. Provisional Application No. 63/175,674, filed Apr. 16, 2021, which is incorporated herein by reference in its entirety.

### REFERENCE TO SEQUENCE LISTING

This application is being filed electronically via EFS-Web and includes an electronically submitted sequence listing in .xml format. The .xml file contains a sequence listing entitled “TIZI-029_D01US_SeqList_ST26-updated.xml” created on Oct. 15, 2025 and having a size of 73,845 bytes. The sequence listing contained in this .xml file is part of the specification and is incorporated herein by reference in its entirety.

### TECHNICAL FIELD

This invention relates generally to methods and use of subcutaneous administration of anti-CD3 monoclonal antibodies (mAbs) either alone or in combination with monoclonal antibodies, that recognize the Interleukin-6 (IL-6) and IL-6 receptor (IL-6R) complex. These mAbs are administered by subcutaneous injection and/or intravenously to patients with an inflammatory, autoimmune or oncological disease such as for example, diabetes, non-alcoholic steatohepatitis (NASH), cancer, and rheumatoid arthritis

### BACKGROUND

Most human cells utilize glucose as the primary substrate, cellular uptake requiring insulin. Insulin signaling is therefore critical for maintaining sugar homeostasis in blood. However, decreases in insulin sensitivity due to the disruption of various molecular pathways causes insulin resistance (IR). IR underpins many metabolic disorders such as type 2 diabetes mellitus (T2DM) and metabolic diseases such as non-alcoholic fatty liver disease (NAFLD), non-alcoholic steatohepatitis (NASH). Although the exact underlying cause of IR has not been fully elucidated, several major mechanisms, including oxidative stress, inflammation, insulin receptor mutations, endoplasmic reticulum stress, and mitochondrial dysfunction have been suggested.

Accordingly, there exists a need for therapies that neutralize the biological activities CD3 to treat and prevent inflammatory, autoimmune and oncological disease such as for example, diabetes, non-alcoholic steatohepatitis (NASH), cancers and rheumatoid arthritis.

### SUMMARY

The disclosure provides a method of treating, preventing or alleviating a symptom of an inflammatory disease, an autoimmune disease or an oncological disease in a subject in need thereof comprising subcutaneously administering to the subject a composition comprising an anti-CD3 antibody. In some embodiments, the antibody is a monoclonal antibody. In some embodiments, the antibody is fully human or humanized.

In some embodiments, the anti-CD3 antibody comprises a heavy chain complementarity determining region 1 (CDRH1) comprising the amino acid sequence GYGMH (SEQ ID NO: 42), a heavy chain complementarity determining region 2 (CDRH2) comprising the amino acid sequence VIWYDGSKKYYVDSVKG (SEQ ID NO: 43), a heavy chain complementarity determining region 3 (CDRH3) comprising the amino acid sequence QMGYWHFDL (SEQ ID NO: 44), a light chain complementarity determining region 1 (CDRL1) comprising the amino acid sequence RASQSVSSYLA (SEQ ID NO: 45), a light chain complementarity determining region 2 (CDRL2) comprising the amino acid sequence DASNRAT (SEQ ID NO: 46), and a light chain complementarity determining region 3 (CDRL3) comprising the amino acid sequence QQRSNWPPLT (SEQ ID NO: 47).

In some embodiments, the anti-CD3 antibody comprises a variable heavy chain amino acid sequence comprising the amino acid sequence of SEQ ID NO: 48 and a variable light chain amino acid sequence comprising the amino acid sequence of SEQ ID NO: 49.

In some embodiments, the anti-CD3 antibody comprises a heavy chain amino acid sequence comprising the amino acid sequence of SEQ ID NO: 50 and a light chain amino acid sequence comprising the amino acid sequence of SEQ ID NO: 51.

In some embodiments, the autoimmune disease is diabetes or rheumatoid arthritis. In some embodiments, the diabetes is Type 1 diabetes. In some embodiments, the autoimmune disease is lupus. In some embodiments, the autoimmune disease is multiple sclerosis.

In some embodiments, the inflammatory disease is Type II diabetes, non-alcoholic fatty liver disease (NAFLD) or non-alcoholic steatohepatitis (NASH). In some embodiments, the oncological disease is hematological cancer. In some embodiments, the hematological cancers is multiple myeloma. In some embodiments, the method further comprises a second administration of the anti-CD3 antibody, wherein the second administration is intranasal. In some embodiments, the method further comprises a second administration of the anti-CD3 antibody, wherein the second administration is subcutaneous and to a different site of the body than the first administration.

In some embodiments, the methods provided further comprise administering an antibody that recognizes the Interleukin-6 (IL-6) and IL-6 receptor (IL-6R) complex (IL-6Rc). In some embodiments, the IL-6 Rc antibody comprising a VH CDR1 region comprising the amino acid sequence of SEQ ID NO: 15, a VH CDR2 region comprising the amino acid sequence of SEQ ID NO: 33, a VH CDR3 region comprising the amino acid sequence of SEQ ID NO: 36, a VL CDR1 region comprising the amino acid sequence of SEQ ID NO: 24, a VL CDR2 region comprising the amino acid sequence of SEQ ID NO: 25, and a VL CDR3 region comprising the amino acid sequence of SEQ ID NO: 26. In some embodiments, the IL-6R antibody is Actemra® or Kevzera®. In some embodiments, the antibody is a monoclonal antibody. In some embodiments, the antibody is fully human.

In some embodiments, the anti-CD3 is administered at a daily dose of 1 mg to 5 mg. In some embodiments, the daily dose is administered once daily. In some embodiments, the daily dose is administered for at least 5 consecutive days.

In some embodiments, the anti-IL-6Rc is administered at a daily dose of 50 mg to 105 mg. In some embodiments, the daily dose is administered once daily. In some embodiments, the daily dose is administered once every 14 days. In some embodiments, the anti-IL-6Rc is administered subcutaneously.

In another aspect, provided herein is a method of treating, preventing or alleviating a symptom of a pulmonary disease in a subject in need thereof comprising administering to the subject a first and a second dose of an antibody that recognizes the Interleukin-6 (IL-6) and IL-6 receptor (IL-6R) complex (IL-6Rc). In some embodiments, the IL-6 Rc antibody comprising a VH CDR1 region comprising the amino acid sequence of SEQ ID NO: 15, a VH CDR2 region comprising the amino acid sequence of SEQ ID NO: 33, a VH CDR3 region comprising the amino acid sequence of SEQ ID NO: 36, a VL CDR1 region comprising the amino acid sequence of SEQ ID NO: 24, a VL CDR2 region comprising the amino acid sequence of SEQ ID NO: 25, and a VL CDR3 region comprising the amino acid sequence of SEQ ID NO: 26. In some embodiments, the first administration is by inhalation and the second administration is by subcutaneous injection. In some embodiments, the first administration is by subcutaneous injection and the second administration is by inhalation. In some embodiments, the pulmonary disease is a pulmonary inflammatory disease. In some embodiments, the pulmonary inflammatory disease is acute respiratory distress syndrome, Idiopathic pulmonary fibrosis (IPF), or systemic pulmonary sclerosis. In some embodiments, the subject has a disease or pathology associated with coronavirus infection.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1 is a diagram illustrating cis and trans IL-6 signaling, and the mechanism of inhibiting both types of IL-6 signaling mediating by the binding of an exemplary antibody.

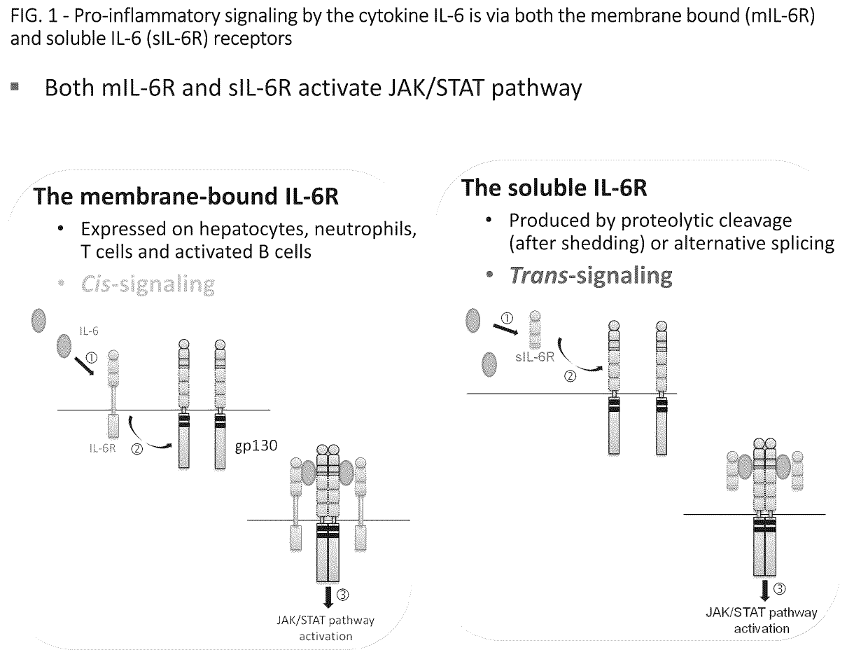

FIG. 2 is a chart comparing features of the IL-6 signaling targeting antibody, N1-1201 (TZLS-501), to approved IL-6 signaling targeting antibodies Actemra and Kevzara.

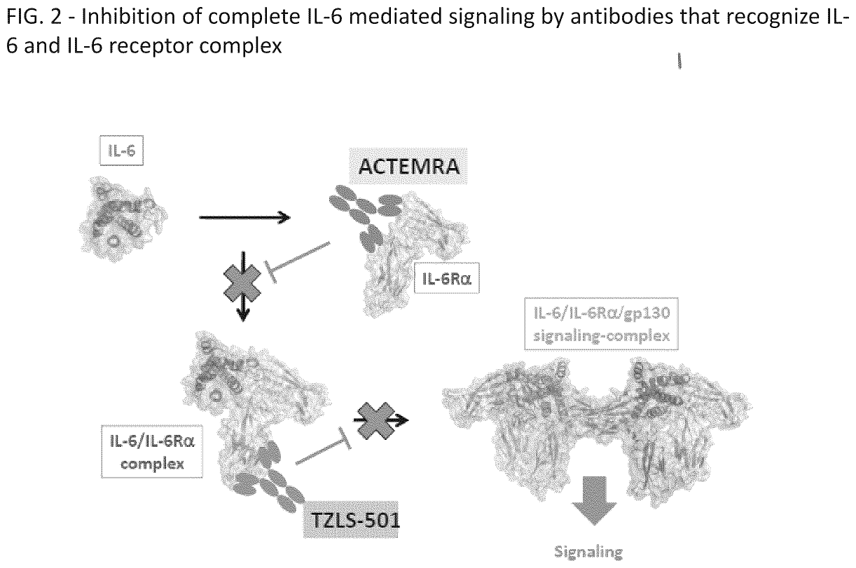

FIG. 3 is a drawing comparing the pharmacokinetics of Foralumab administered subcutaneously (S.C) at 1× and 2× doses and intravenously (IV).

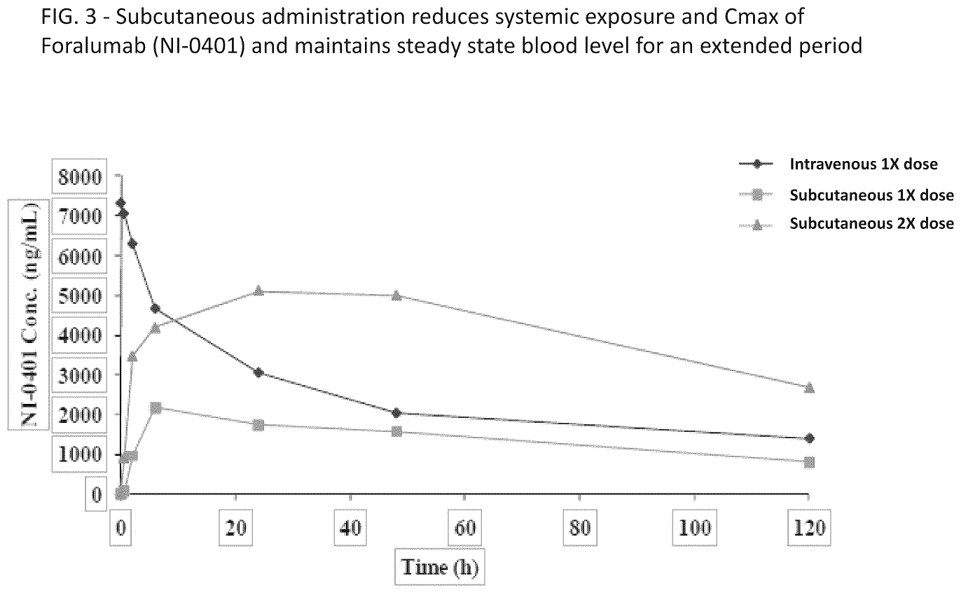

FIG. 4 is drawing showing the dosing regimen for combination of Foralumab (TZLS-401, formerly Novimmune NI-0401; anti-human CD3) with TZLS-501 (NI-1201) anti-IL-6/IL-6R antibody administered subcutaneously for treatment of autoimmune and inflammatory indications.

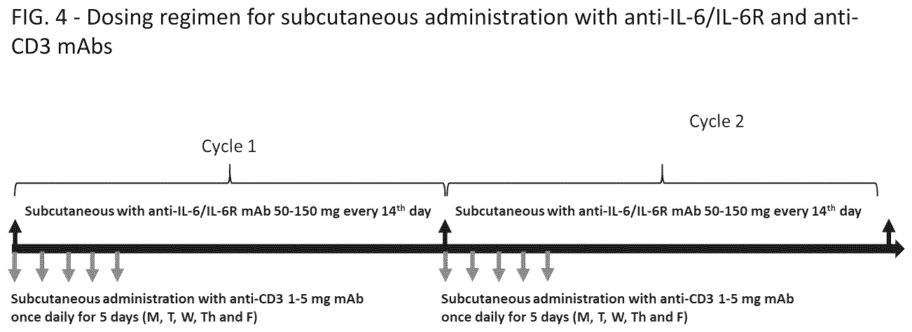

FIG. 5 is a chart showing foralumab concentration in plasma post single administration of foralumab either IV or SC at a dose of 0.3 mg/kg IV (IV 1×), 0.3 mg/kg SC (SC 1×) or 0.6 mg/kg SC (SC 2×). A total of 6 mice per time points were analyzed in each group. Data are presented as mean values of foralumab concentration+/−SEM. The table (right panel) summarizes the mean foralumab concentration obtained in plasma (in ng/ml) for each group and each time points.

FIG. 6 is a chart showing PK profiles in male vs female mice administrated with foralumab either IV at 0.3 mg/kg (IVIx) or SC at a dose of 0.3 mg/kg (SC1×) or 0.6 mg/kg (SC2×). A total of 3 males and 3 females mice per time points were analyzed in each group. Data are provided in ng/ml and presented as mean values+/−SEM.

FIG. 7 is a chart showing percentage of human CD3ε modulation at the cell surface of blood CD4+ T cells in LCD3 mice which received foralumab either IV at 0.3 mg/kg (IV1×) or SC at a dose of 0.3 mg/kg (SC1×) or 0.6 mg/kg (SC2×). A total of 6 mice per time point were analyzed in each group. Data are provided as the percentage of human CD3 modulation, determined from the mean values using the placebo control (PBS injected IV) as reference. Data are presented as mean values+/−SEM. The table (lower panel) summarizes the mean percentage of human CD3 modulation obtained for each group and each time points

FIG. 8 is a chart showing CD4+ T cell count in blood post administration of foralumab either IV at 0.3 mg/kg (IVIx) or SC at a dose of 0.3 mg/kg (SC1×) or 0.6 mg/kg (SC2×). A total of 6 mice per time point were analyzed in each group. Data are provided as the absolute number of CD4+ T cells/μl of blood and presented as mean values+/−SEM. The table (lower panel) summarizes the mean absolute values of CD4+ T cells/μl of blood for each group and each time point.

FIG. 9 is a chart showing CD8+ T cell count in blood post administration of foralumab either IV at 0.3 mg/kg (IVIx) or SC at a dose of 0.3 mg/kg (SC1×) or 0.6 mg/kg (SC2×). A total of 6 mice per time point were analyzed in each group. Data are provided as the absolute number of CD8+ T cells/μl of blood and presented as mean values+/−SEM. The table (lower panel) summarizes the mean absolute values of CD8+ T cells/μl of blood for each group and each time point.

### DETAILED DESCRIPTION

The present invention provides monoclonal antibodies that specifically bind the human CD3 for the treatment, prevention or alleviating a symptom of an inflammatory, autoimmune or oncological disease. Exemplary diseases suitable for the methods of the invention include for example diabetes (Type 1 and Type 2), non-alcoholic fatty liver disease (NAFLD), non-alcoholic steatohepatitis (NASH), cachexia, Rheumatoid Arthritis, cancers including hematological cancers, such as multiple myeloma. These antibodies are collectively referred to herein as “huCD3” antibodies. The antibody is e.g., a fully human antibody.

The inventors have shown that that the subcutaneous administration of an anti-CD3 monoclonal antibody is immunomodulatory and suppresses hyperactive immune responses in a large number of inflammatory and autoimmune disease. The mechanism of action includes the induction of regulatory T cells, downregulation of Th1 and Th17 cells, and downregulation of CD8 cells.

Furthermore, the inventors have shown a fully humanized anti-CD3 Mab, foralumab administered subcutaneously maintains a steady state blood level for an extended period of time in contrast to intravenous administration. Thus, the subcutaneous administration of foralumab has immunomodulatory properties that would be beneficial to treat the immune response that occurs with inflammatory, autoimmune and oncological disease.

Accordingly, in various aspects the invention provides method for treating inflammatory, autoimmune and oncological disease by administering anti-CD3 mAbs either alone or in combination with monoclonal antibodies that recognize the Interleukin-6 (IL-6) and IL-6 receptor (IL-6R) complex ((“IL-6Rc”). The treatment may be administered a) anti-CD3 given subcutaneously; b) anti-CD3 given intravenously; c) anti-CD3 given combination of subcutaneously and intravenously; d) anti-CD3 given subcutaneously or intravenously in combination with anti-IL-6Rc; e) anti-CD3 given subcutaneously and intravenously in combination with anti-IL-6Rc; f) anti-CD3 given subcutaneously and intranasally; g) anti-CD3 given subcutaneously and intranasally in combination with anti-IL-6Rc; h) anti-IL-6Rc given subcutaneously and by inhalation; and i) anti-IL-6Rc given subcutaneously and by inhalation in combination with anti-CD3.

The delivery of antibodies subcutaneously is advantageous as same level of efficacy can be achieved at a much lower dose and side effects are also minimized as compared to other routes of administration.

### CD3 Antibodies

The anti-CD3 antibodies can be any antibodies specific for CD3. The anti-CD3 antibody can be a polyclonal, monoclonal, recombinant, e.g., a chimeric, de-immunized or humanized, fully human, non-human, e.g., murine, single chain antibody or single domain antibody. In some embodiments the antibody has effector function and can fix complement. In some embodiments, the antibody has reduced or no ability to bind an Fc receptor. For example, the anti-CD3 antibody can be an isotype or subtype, fragment or other mutant, which does not support binding to an Fc receptor, e.g., it has a mutagenized or deleted Fc receptor binding region. The antibody can be coupled to a toxin or imaging agent.

A number of anti-CD3 antibodies are known, including but not limited to OKT3 (muromonab/Orthoclone OKT3™, Ortho Biotech, Raritan, N.J.; U.S. Pat. No. 4,361,549); hOKT3(1 (Herold et al., N.E.J.M. 346(22):1692-1698 (2002); HuM291 (Nuvion™, Protein Design Labs, Fremont, Calif); gOKT3-5 (Alegre et al., J. Immunol. 148(11):3461-8 (1992); 1F4 (Tanaka et al., J. Immunol. 142:2791-2795 (1989)); G4.18 (Nicolls et al., Transplantation 55:459-468 (1993)); 145-2C11 (Davignon et al., J. Immunol. 141(6):1848-54 (1988)); and as described in Frenken et al., Transplantation 51(4):881-7 (1991); U.S. Pat. Nos. 6,491,9116, 6,406,696, and 6,143,297).

Methods for making such antibodies are also known. A full-length CD3 protein or antigenic peptide fragment of CD3 can be used as an immunogen, or can be used to identify anti-CD3 antibodies made with other immunogens, e.g., cells, membrane preparations, and the like, e.g., E rosette positive purified normal human peripheral T cells, as described in U.S. Pat. Nos. 4,361,549 and 4,654,210. The anti-CD3 antibody can bind an epitope on any domain or region on CD3.

Chimeric, humanized, de-immunized, or completely human antibodies are desirable for applications which include repeated administration, e.g., therapeutic treatment of human subjects.

Chimeric antibodies contain portions of two different antibodies, typically of two different species. Generally, such antibodies contain human constant regions and variable regions from another species, e.g., murine variable regions. For example, mouse/human chimeric antibodies have been reported which exhibit binding characteristics of the parental mouse antibody, and effector functions associated with the human constant region. See, e.g., Cabilly et al., U.S. Pat. No. 4,816,567; Shoemaker et al., U.S. Pat. No. 4,978,745; Beavers et al., U.S. Pat. No. 4,975,369; and Boss et al., U.S. Pat. No. 4,816,397, all of which are incorporated by reference herein. Generally, these chimeric antibodies are constructed by preparing a genomic gene library from DNA extracted from pre-existing murine hybridomas (Nishimura et al., Cancer Research, 47:999 (1987)). The library is then screened for variable region genes from both heavy and light chains exhibiting the correct antibody fragment rearrangement patterns. Alternatively, cDNA libraries are prepared from RNA extracted from the hybridomas and screened, or the variable regions are obtained by polymerase chain reaction. The cloned variable region genes are then ligated into an expression vector containing cloned cassettes of the appropriate heavy or light chain human constant region gene. The chimeric genes can then be expressed in a cell line of choice, e.g., a murine myeloma line. Such chimeric antibodies have been used in human therapy.

Humanized antibodies are known in the art. Typically, “humanization” results in an antibody that is less immunogenic, with complete retention of the antigen-binding properties of the original molecule. In order to retain all the antigen-binding properties of the original antibody, the structure of its combining-site has to be faithfully reproduced in the “humanized” version. This can potentially be achieved by transplanting the combining site of the nonhuman antibody onto a human framework, either (a) by grafting the entire nonhuman variable domains onto human constant regions to generate a chimeric antibody (Morrison et al., Proc. Natl. Acad. Sci., USA 81:6801 (1984); Morrison and Oi, Adv. Immunol. 44:65 (1988) (which preserves the ligand-binding properties, but which also retains the immunogenicity of the nonhuman variable domains); (b) by grafting only the nonhuman CDRs onto human framework and constant regions with or without retention of critical framework residues (Jones et al. Nature, 321:522 (1986); Verhoeyen et al., Science 239:1539 (1988)); or (c) by transplanting the entire nonhuman variable domains (to preserve ligand-binding properties) but also “cloaking” them with a human-like surface through judicious replacement of exposed residues (to reduce antigenicity) (Padlan, Molec. Immunol. 28:489 (1991)).

Humanization by CDR grafting typically involves transplanting only the CDRs onto human fragment onto human framework and constant regions. Theoretically, this should substantially eliminate immunogenicity (except if allotypic or idiotypic differences exist). However, it has been reported that some framework residues of the original antibody also need to be preserved (Riechmann et al., Nature 332:323 (1988); Queen et al., Proc. Natl. Acad. Sci. USA 86:10, 029 (1989)). The framework residues which need to be preserved can be identified by computer modeling. Alternatively, critical framework residues may potentially be identified by comparing known antibody combining site structures (Padlan, Molec. Immun. 31(3):169-217 (1994)). The invention also includes partially humanized antibodies, in which the 6 CDRs of the heavy and light chains and a limited number of structural amino acids of the murine monoclonal antibody are grafted by recombinant technology to the CDR-depleted human IgG scaffold (Jones et al., Nature 321:522-525 (1986)).

Deimmunized antibodies are made by replacing immunogenic epitopes in the murine variable domains with benign amino acid sequences, resulting in a deimmunized variable domain. The deimmunized variable domains are linked genetically to human IgG constant domains to yield a deimmunized antibody (Biovation, Aberdeen, Scotland).

The anti-CD3 antibody can also be a single chain antibody. A single-chain antibody (scFV) can be engineered (see, for example, Colcher et al., Ann. N. Y. Acad. Sci. 880:263-80 (1999); and Reiter, Clin. Cancer Res. 2:245-52 (1996)). The single chain antibody can be dimerized or multimerized to generate multivalent antibodies having specificities for different epitopes of the same target CD3 protein. In some embodiments, the antibody is monovalent, e.g., as described in Abbs et al., Ther. Immunol. 1(6):325-31 (1994), incorporated herein by reference.

Exemplary anti-CD3 antibodies, comprise a heavy chain complementarity determining region 1 (CDRH1) comprising the amino acid sequence GYGMH (SEQ ID NO: 42), a heavy chain complementarity determining region 2 (CDRH2) comprising the amino acid sequence VIWYDGSKKYYVDSVKG (SEQ ID NO: 43), a heavy chain complementarity determining region 3 (CDRH3) comprising the amino acid sequence QMGYWHFDL (SEQ ID NO: 44), a light chain complementarity determining region 1 (CDRL1) comprising the amino acid sequence RASQSVSSYLA (SEQ ID NO: 45), a light chain complementarity determining region 2 (CDRL2) comprising the amino acid sequence DASNRAT (SEQ ID NO: 46), and a light chain complementarity determining region 3 (CDRL3) comprising the amino acid sequence QQRSNWPPLT (SEQ ID NO: 47).

In some embodiments, the anti-CD3 antibody comprises a variable heavy chain amino acid sequence comprising QVQLVESGGGVVQPGRSLRLSCAASGFKFSGYGMHWVRQAPGKGLEWVAVIWYD GSKKYYVDSVKGRFTISRDNSKNTLYLQMNSLRAEDTAVYYCARQMGYWHFDLW GRGTLVTVSS (SEQ ID NO: 48) and a variable light chain amino acid sequence comprising EIVLTQSPATLSLSPGERATLSCRASQSVSSYLAWYQQKPGQAPRLLIYDASNRATGI PARFSGSGSGTDFTLTISSLEPEDFAVYYCQQRSNWPPLTFGGGTKVEIK (SEQ ID NO: 49).

Preferably, the anti-CD3 antibody comprises a heavy chain amino acid sequence comprising: QVQLVESGGGVVQPGRSLRLSCAASGFKFSGYGMHWVRQAPGKGLEWVAVIWYD GSKKYYVDSVKGRFTISRDNSKNTLYLQMNSLRAEDTAVYYCARQMGYWHFDLW GRGTLVTVSSASTKGPSVFPLAPSSKSTSGGTAALGCLVKDYFPEPVTVSWNSGALTS GVHTFPAVLQSSGLYSLSSVVTVPSSSLGTQTYICNVNHKPSNTKVDKRVEPKSCDK THTCPPCPAPEAEGGPSVFLFPPKPKDTLMISRTPEVTCVVVDVSHEDPEVKFNWYV DGVEVHNAKTKPREEQYNSTYRVVSVLTVLHQDWLNGKEYKCKVSNKALPAPIEK TISKAKGQPREPQVYTLPPSREEMTKNQVSLTCLVKGFYPSDIAVEWESNGQPENNY KTTPPVLDSDGSFFLYSKLTVDKSRWQQGNVFSCSVMHEALHNHYTQKSLSLSPGK (SEQ ID NO: 50) and a light chain amino acid sequence comprising: EIVLTQSPATLSLSPGERATLSCRASQSVSSYLAWYQQKPGQAPRLLIYDASNRATGI PARFSGSGSGTDFTLTISSLEPEDFAVYYCQQRSNWPPLTFGGGTKVEIKRTVAAPSV FIFPPSDEQLKSGTASVVCLLNNFYPREAKVQWKVDNALQSGNSQESVTEQDSKDST YSLSSTLTLSKADYEKHKVYACEVTHQGLSSPVTKSFNRGEC (SEQ ID NO: 51). This anti-CD3 antibody is referred to herein as NI-0401, Foralumab, or 28F11-AE. (See e.g., Dean Y, Dépis F, Kosco-Vilbois M. “Combination therapies in the context of anti-CD3 antibodies for the treatment of autoimmune diseases.” Swiss Med Wkly. (2012) (the contents of which are hereby incorporated by reference in its entirety).

In some embodiments, the anti-CD3 antibody is a fully human antibody or a humanized antibody. In some embodiments, the anti-CD3 antibody formulation includes a full length anti-CD3 antibody. In alternative embodiments, the anti-CD3 antibody formulation includes an antibody fragment that specifically binds CD3. In some embodiments, the anti-CD3 antibody formulation includes a combination of full-length anti-CD3 antibodies and antigen binding fragments that specifically bind CD3.

In some embodiments, the antibody or antigen-binding fragment thereof that binds CD3 is a monoclonal antibody, domain antibody, single chain, Fab fragment, a F(ab′)₂ fragment, a scFv, a scAb, a dAb, a single domain heavy chain antibody, or a single domain light chain antibody. In some embodiments, such an antibody or antigen-binding fragment thereof that binds CD3 is a mouse, other rodent, chimeric, humanized or fully human monoclonal antibody.

Optionally, the anti-CD3 antibody or antigen binding fragment thereof used in the formulations of the disclosure includes at least one amino acid mutation. Typically, the mutation is in the constant region. The mutation results in an antibody that has an altered effector function. An effector function of an antibody is altered by altering, i.e., enhancing or reducing, the affinity of the antibody for an effector molecule such as an Fc receptor or a complement component. For example, the mutation results in an antibody that is capable of reducing cytokine release from a T-cell. For example, the mutation is in the heavy chain at amino acid residue 234, 235, 265, or 297 or combinations thereof. Preferably, the mutation results in an alanine residue at either position 234, 235, 265 or 297, or a glutamate residue at position 235, or a combination thereof.

Preferably, the anti-CD3 antibody provided herein contains one or more mutations that prevent heavy chain constant region-mediated release of one or more cytokine(s) in vivo.

In some embodiments, the anti-CD3 antibody or antigen binding fragment thereof used in the formulations of the disclosure is a fully human antibody. The fully human CD3 antibodies used herein include, for example, a L234A and L235E mutation in the Fc region, such that cytokine release upon exposure to the anti-CD3 antibody is significantly reduced or eliminated. The L234A and L235E mutation in the Fc region of the anti-CD3 antibodies provided herein reduces or eliminates cytokine release when the anti-CD3 antibodies are exposed to human leukocytes, whereas the mutations described below maintain significant cytokine release capacity. For example, a significant reduction in cytokine release is defined by comparing the release of cytokines upon exposure to the anti-CD3 antibody having an L234A and L235E mutation in the Fc region to level of cytokine release upon exposure to another anti-CD3 antibody having one or more of the mutations described below. Other mutations in the Fc region include, for example, L234A and L235A, L235E, N297A, D265A, or combinations thereof.

### IL-6/IL-6 Receptor Complex Antibodies

Exemplary IL-6/IL-6 receptor complex (IL-6Rc) antibodies useful in the compositions and methods of the disclosure include, for example, Actemra® (tocilizumab), or Kevzera® (sarilumab).

Other, IL-6Rc antibodies include the 39B9 VL1 antibody, the 39B9 VL5 antibody, the 12A antibody, and the 5C antibody described in WO/2009/140348, the contents of which are hereby incorporated by reference in its entirety. These antibodies show specificity for human IL-6Rc and/or both IL-6Rc and IL-6R and they have been shown to inhibit the functional activity of IL-6Rc (i.e., binding to gp130 to induce the signaling cascade) in vitro.

In some embodiments, the anti-6Rc antibody comprises a light chain and a heavy chain sequence. In some embodiments, the anti-6Rc antibody light chain sequence is SEQ ID NO: 53. In some embodiments, the anti-6Rc antibody heavy chain sequence is SEQ ID NO: 52. In some embodiments, the anti-6Rc antibody comprises SEQ ID NO: 53 and SEQ ID NO: 52.

The 39B9 VL1 and 39B9 VL5 antibodies share a common heavy chain variable region (SEQ ID NO:2) encoded by the nucleic acid sequence shown in SEQ ID NO: 1. The 39B9 VL1 antibody includes a light chain variable region (SEQ ID NO: 4) encoded by the nucleic acid sequence shown in SEQ ID NO: 3. The 39B9 VL5 antibody includes a light chain variable region (SEQ ID NO: 6) encoded by the nucleic acid sequence shown in SEQ ID NO: 5. The 12A antibody includes a heavy chain variable region (SEQ ID NO:8) encoded by the nucleic acid sequence shown in SEQ ID NO: 7. The 12A antibody includes a light chain variable region (SEQ ID NO: 10) encoded by the nucleic acid sequence shown in SEQ ID NO: 9. The 5C antibody includes a heavy chain variable region (SEQ ID NO: 12) encoded by the nucleic acid sequence shown in SEQ ID NO: 11. The 5C antibody includes a light chain variable region (SEQ ID NO: 14) encoded by the nucleic acid sequence shown in SEQ ID NO: 13.

TABLE 1
Illustrative IL-6/IL-6Rc Antibody Sequences

| | SEQ ID | |
|---|---|---|
| Name | NO. | Sequence |
| 3B9 VL1-VH | 1 | CAGGTGCAGCTGGTGCAGTCTGGGGCTGAGGTGAA |
| nucleic acid | | GAAGCCTGGGTCCTCGGTGAAGGTCTCCTGCAAGG |
| sequence | | CTTCTGGAGGCACCTTCAGCAGCTATGCTATCAGCT |
| | | GGGTGCGCCAGGCCCCTGGACAAGGGCTTGAGTGG |
| | | ATGGGAGGGATCATCCCTCTCTTTGATACAACAAAG |
| | | TACGCACAGCAGTTCCAGGGCAGAGTCACGATTAC |
| | | CGCGGACGAATCCACGAGCACAGCCTACATGGAGC |
| | | TGAGCAGCCTGAGATCTGAGGACACGGCCGTATTTT |
| | | ACTGTGCGAGAGATCGGGATATTTTGACTGATTATT |
| | | ATCCCATGGGCGGTATGGACGTCTGGGGCCAAGGG |
| | | ACCACGGTCACCGTCTCCTCA |
| 3B9 VL1-VH | 2 | QVQLVQSGAEVKKPGSSVKVSCKASGGTFSSYAISWV |
| amino acid | | RQAPGQGLEWMGGIIPLFDTTKYAQQFQGRVTITADE |
| sequence | | STSTAYMELSSLRSEDTAVFYCARDRDILTDYYPMGG |
| | | MDVWGQGTTVTVSS |
| 39B9 VL1-VL | 3 | GCCATCCAGTTGACCCAGTCTCCATCCTCCCTGTCT |
| nucleic acid | | GCATCTGTAGGAGACAGAGTCACCATCACTTGCCG |
| sequence | | GGCAAGTCAGGGCATTAGCAGTGTTTTAGCCTGGTA |
| | | TCAGCAGAAACCAGGGAAAGCTCCTAAGCTCCTGA |
| | | TCTATGATGCCTCCAGTTTGGAAAGTGGGGTCCCAT |
| | | CAAGGTTCAGCGGCAGTGGATCTGGGACAGATTTC |
| | | ACTCTCACCATCAGCAGCCTGCAGCCTGAAGATTTT |
| | | GCAACTTATTACTGTCAACAGTCTAATAGTTACCCG |
| | | CTCACTTTCGGCGGAGGGACCAAGGTGGAGATCAA |
| | | ACGT |
| 39B9 VL1-VL | 4 | AIQLTQSPSSLSASVGDRVTITCRASQGISSVLAWYQQ |
| amino acid | | KPGKAPKLLIYDASSLESGVPSRFSGSGSGTDFTLTISS |
| sequence | | LQPEDFATYYCQQSNSYPLTFGGGTKVEIKR |
| 39B9 VL5-VL | 5 | GACATCCTGATGACCCAGTCTCCATCCTCCCTGTCT |
| nucleic acid | | GCATCTGTAGGAGACAGAGTCACCATCACTTGTCGG |
| sequence | | GCGAGTCAGGATATTAGCAGCTGGTTAGCCTGGTAT |
| | | CAGCAGAAACCAGGGAAAGCTCCTAAGCTCCTGAT |
| | | CTATGATGCCTCCAGTTTGGAAAGTGGGGTCCCATC |
| | | AAGGTTCAGCGGCAGTGGATCTGGGACAGATTTCA |
| | | CTCTCACCATCAGCAGCCTGCAGCCTGAAGATTTTG |
| | | CAACTTATTACTGTCAACAGTCTAATAGTTACCCGC |
| | | TCACTTTCGGCGGAGGGACCAAGGTGGAGATCAAA |
| | | CGA |
| 39B9 VL5-VL | 6 | DILMTQSPSSLSASVGDRVTITCRASQDISSWLAWYQQ |
| amino acid | | KPGKAPKLLIYDASSLESGVPSRFSGSGSGTDFTLTISS |
| sequence | | LQPEDFATYYCQQSNSYPLTFGGGTKVEIKR |
| 12A VH nucleic | 7 | CAGGTGCAGCTGGTGGAGTCTTGGGGAGGCGTGGT |
| acid sequence | | CCAGCCTGGGAGGTCCCTGAGACTCTCCTGTGCAGC |
| | | GTCTGGATTCACCTTCAGTAACTATGACATGTACTG |
| | | GGTCCGCCAGGCTCCAGGCAAGGGGCTGGAGTGGG |
| | | TGGCAGTTATATTAGATGATGGAAATAATAATTACT |
| | | ACGCAGACTCCGTGAAGGGCCGATTCACCATCTCCA |
| | | GAGACAATTCCAAGAAAAAGGTGTATCTGCAAATG |
| | | AATAGCCTGAGAGCTGAGGACACGGCTGTGTATTA |
| | | CTGTGTGAGAGCGTCCCCTAACTGGGGTCTTCTTGA |
| | | CTTCTGGGGCCAGGGAACCCTGGTCACCGTCTCGAG |
| | | T |
| 12A VH amino acid | 8 | QVQLVESWGGVVQPGRSLRLSCAASGFTFSNYDMYW |
| sequence | | VRQAPGKGLEWVAVILDDGNNNYYADSVKGRFTISR |
| | | DNSKKKVYLQMNSLRAEDTAVYYCVRASPNWGLLD |
| | | FWGQGTLVTVSS |
| 12A VL nucleic | 9 | GAAATTGTGTTGACACAGTCTCCATCCTCACTGTCT |
| acid sequence | | GCATCTGTAGGAGACAGAGTCACCATCACTTGTCGG |
| | | GCGAGTCAGGGTATTAGCAGCTGGTTAGCCTGGTAT |
| | | CAGCAGAAACCAGGGAAAGCTCCTAAGCTCCTGAT |
| | | CTATGATGCCTCCAGTTTGGAAAGTGGGGTCCCATC |
| | | AAGGTTCAGCGGCAGTGGATCTGGGACAGATTTCA |
| | | CTCTCACCATCAGCAGCCTGCAGCCTGAAGATTTTG |
| | | CAACTTATTACTGTCAACAGTTTAATAGTTACCCGA |
| | | TCACCTTCGGCCAAGGGACACGACTGGAGATTAAA |
| | | CGT |
| 12A VL amino acid | 10 | EIVLTQSPSSLSASVGDRVTITCRASQGISSWLAWYQQ |
| sequence | | KPGKAPKLLIYDASSLESGVPSRFSGSGSGTDFTLTISS |
| | | LQPEDFATYYCQQFNSYPITFGQGTRLEIKR |
| 5C VH nucleic acid | 11 | CAGGTGCAGCTGGTGCAGTCTGGGGGAGGCGTGGT |
| sequence | | CCAGCCTGGGAGGTCCCTGAGACTCTCCTGTGCAGC |
| | | CTCTGGATTCATCTTCAGTAGCTATGACATGTACTG |
| | | GGTCCGCCAGGCTCCAGGCAAGGGGCTGGAGTGGG |
| | | TGGCAGTTATATTATATGATGGAAATAATAAATACT |
| | | ACGCAGACTCCGTGAAGGGCCGATTCACCATCTCCA |
| | | GAGACAATTCCAAGAACACGGTGTATCTGCAAATG |
| | | AACAGCCTGAGAGCTGAGGACACGGCTGTGTATTA |
| | | CTGTGTGAGAGCGTCCCCTAACTGGGGTCTTTTTGA |
| | | CTTCTGGGGCCAGGGAACCCTGGTCACCGTCTCGAG |
| | | T |
| 5C VH amino acid | 12 | QVQLVQSGGGVVQPGRSLRLSCAASGFIFSSYDMYW |
| sequence | | VRQAPGKGLEWVAVILYDGNNKYYADSVKGRFTISR |
| | | DNSKNTVYLQMNSLRAEDTAVYYCVRASPNWGLFDF |
| | | WGQGTLVTVSS |
| 5C VL nucleic acid | 13 | GACATCCAGATGACCCAGTCTCCATCCTCCCTGTCT |
| sequence | | GCATCTGTAGGAGACAGAGTCACCATCACTTGCCG |
| | | GGCAAGTCAGGGCATTAGCAGTGATTTAGCCTGGT |
| | | ATCAGCAGAAACCAGGGAAAGCTCCTAAGCTCCTG |
| | | ATGTATGATGCCTCCAGTTTGGAAAGTGGGGTCCCA |
| | | TCAAGGTTCAGCGGCAGTGGATCTGGGACAGATTTC |
| | | ACTCTCACCATCAGCAGCCTGCAGCCTGAAGATTTT |
| | | GCAACTTATTACTGTCAACAGTTTAATAGTTACCCG |
| | | ATCACCTTCGGCCAAGGGACACGACTGGAGATTAA |
| | | ACGT |
| 5C VL amino acid | 14 | DIQMTQSPSSLSASVGDRVTITCRASQGISSDLAWYQQ |
| sequence | | KPGKAPKLLMYDASSLESGVPSRFSGSGSGTDFTLTIS |
| | | SLQPEDFATYYCQQFNSYPITFGQGTRLEIKR |

hulL-6Rc antibodies of the invention additionally comprise, for example, the heavy chain complementarity determining regions (VH CDRs) shown below in Table 2, the light chain complementarity determining regions (VL CDRs) shown in Table 3, and combinations thereof.

TABLE 2
VH CDR sequences from antibody clones that
bind and neutralize IL-6Rc biological activity

| Clone | | | |
|---|---|---|---|
| Name | VH CDR1 | VH CDR2 | VH CDR3 |
| 39B9 | SYAIS | GIIPLFDTTKYAQQF | CARDRDILTDYYPMG |
| | (SEQ ID | QG | GMDV |
| | NO: 15) | (SEQ ID NO 16) | (SEQ ID NO: 17) |
| 12A | NYDMY | VILDDGNNNYYADSV | CVRASPNWGLLDF |
| | (SEQ ID | KG | (SEQ ID NO: 20) |
| | NO: 18) | (SEQ ID NO: 19) | |
| 5C | SYDMY | VILYDGNNKYYADSV | CVRASPNWGLFDF |
| | (SEQ ID | KG | (SEQ ID NO: 23) |
| | NO: 21) | (SEQ ID NO: 22) | |

TABLE 3
VL CDR sequences from antibody clones
that bind and neutralize IL-6Rc

| Clone | | | |
|---|---|---|---|
| Name | VL CDR1 | VL CDR2 | VL CDR3 |
| 39B9 VL1 | RASQGISSVLA | DASSLES | QQSNSYPLT |
| | (SEQ ID NO: 24) | (SEQ ID | (SEQ ID NO: 26) |
| | | NO: 25) | |
| 39B9 VL5 | RASQDISSWLA | DASSLES | QQSNSYPLT |
| | (SEQ ID NO: 27) | (SEQ ID | (SEQ ID NO: 26) |
| | | NO: 25) | |
| 12A | RASQGISSWLA | DASSLES | QQSNSYPIT |
| | (SEQ ID NO: 28) | (SEQ ID | (SEQ ID NO: 29) |
| | | NO: 25) | |
| 5C | RASQGISSVDA | DASSLES | QQSNSYPIT |
| | (SEQ ID NO: 30) | (SEQ ID | (SEQ ID NO: 29) |
| | | NO: 25) | |

The hulL-6Rc antibodies of the invention serve to modulate, block, inhibit, reduce, antagonize, neutralize or otherwise interfere with the functional activity of IL-6Rc. Functional activities of IL-6Rc include for example, intracellular signaling via activation of the JAK/STAT pathway and activation of the MAPK cascade, acute phase protein production, antibody production and cellular differentiation and/or proliferation. For example, the hulL-6Rc antibodies completely or partially inhibit IL-6Rc functional activity by partially or completely modulating, blocking, inhibiting, reducing antagonizing, neutralizing, or otherwise interfering with the binding of IL-6Rc to the signal-transducing receptor component gp130.

The huIL-6Rc antibodies are considered to completely modulate, block, inhibit, reduce, antagonize, neutralize or otherwise interfere with IL-6Rc functional activity when the level of IL-6Rc functional activity in the presence of the huIL-6Rc antibody is decreased by at least 95%, e.g., by 96%, 97%, 98%, 99% or 100% as compared to the level of IL-6Rc functional activity in the absence of binding with a huIL-6Rc antibody described herein. The huIL-6Rc antibodies are considered to partially modulate, block, inhibit, reduce, antagonize, neutralize or otherwise interfere with IL-6Rc functional activity when the level of IL-6Rc activity in the presence of the huIL-6Rc antibody is decreased by less than 95%, e.g., 10%, 20%, 25%, 30%, 40%, 50%, 60%, 75%, 80%, 85% or 90% as compared to the level of IL-6Rc activity in the absence of binding with a huIL-6Rc antibody described herein.

Variants of huIL-6Rc Antibodies

Variants of the huIL-6Rc antibodies are made using any of a variety of art-recognized techniques. For example, variant huIL-6Rc antibodies include antibodies having one or more amino acid modifications, such as, for example, an amino acid substitution, at position within the antibody sequence.

Preferred locations for amino acid substitutions are shown as bold, underlined residues below in Table 4. The amino acid residues in bold/underline can be replaced with any amino acid residue. In preferred embodiments, the amino acid residues in bold/underline are replaced with the amino acid residues shown below in Table 4. In these embodiments, the antibody comprises (i) the consensus amino acid sequence QQSXSYPLT (SEQ ID NO: 31) in the light chain complementarity determining region 3 (CDR3), where X is N or Q; (ii) the consensus amino acid sequence GIIPX1FX2TTKYAQX3FQG (SEQ ID NO: 32) in the heavy chain complementarity determining region 2 (CDR2), where X1 is L or A, X2 is D or E, and X3 is Q or K; (iii) the consensus amino acid sequence DRDILTDYYPXGGMDV (SEQ ID NO: 33) in the heavy chain complementarity determining region 3 (CDR3), where X is M or L; and (iv) the consensus amino acid sequence TAVXYCAR (SEQ ID NO: 34) in the framework region 3 (FRW3), where X is F or Y.

The NI-1201-wild type (NI-1201-WT) antibody listed in Table 4 comprises the amino acid sequence QQSNSYPLT (SEQ ID NO: 26) in the light chain CDR3 region, the amino acid sequence GIIPLFDTTKYAQQFQG (SEQ ID NO: 16) in the heavy chain CDR2 region, the amino acid sequence DRDILTDYYPMGGMDV (SEQ ID NO: 35) in the heavy chain CDR3 region, and the amino acid sequence TAVFYCAR (SEQ ID NO: 36) in the FRW3 region.

The NI-1201-A antibody listed in Table 4 comprises the amino acid sequence QQSNSYPLT (SEQ ID NO: 26) in the light chain CDR3 region, the amino acid sequence GIIPLFDTTKYAQKFQG (SEQ ID NO: 37) in the heavy chain CDR2 region, the amino acid sequence DRDILTDYYPMGGMDV (SEQ ID NO: 35) in the heavy chain CDR3 region, and the amino acid sequence TAVYYCAR (SEQ ID NO: 38) in the FRW3 region.

The NI-1201-B antibody listed in Table 4 comprises the amino acid sequence QQSNSYPLT (SEQ ID NO: 26) in the light chain CDR3 region, the amino acid sequence GIIPLFDTTKYAQKFQG (SEQ ID NO: 37) in the heavy chain CDR2 region, the amino acid sequence DRDILTDYYPLGGMDV (SEQ ID NO: 39) in the heavy chain CDR3 region, and the amino acid sequence TAVYYCAR (SEQ ID NO: 38) in the FRW3 region.

The NI-1201-C antibody listed in Table 4 comprises the amino acid sequence QQSNSYPLT (SEQ ID NO: 26) in the light chain CDR3 region, the amino acid sequence GIIPAFETTKYAQKFQG (SEQ ID NO: 40) in the heavy chain CDR2 region, the amino acid sequence DRDILTDYYPLGGMDV (SEQ ID NO: 39) in the heavy chain CDR3 region, and the amino acid sequence TAVYYCAR (SEQ ID NO: 38) in the FRW3 region.

The NI-1201-D antibody listed in Table 4 comprises the amino acid sequence QQSQSYPLT (SEQ ID NO: 41) in the light chain CDR3 region, the amino acid sequence GIIPAFETTKYAQKFQG (SEQ ID NO: 40) in the heavy chain CDR2 region, the amino acid sequence DRDILTDYYPLGGMDV (SEQ ID NO: 39) in the heavy chain CDR3 region, and the amino acid sequence TAVYYCAR (SEQ ID NO: 38) in the FRW3 region.

The NI-1201-E antibody listed in Table 4 comprises the amino acid sequence QQSQSYPLT (SEQ ID NO: 41) in the light chain CDR3 region, the amino acid sequence GIIPLFDTTKYAQKFQG (SEQ ID NO: 37) in the heavy chain CDR2 region, the amino acid sequence DRDILTDYYPLGGMDV (SEQ ID NO: 39) in the heavy chain CDR3 region, and the amino acid sequence TAVYYCAR (SEQ ID NO: 38) in the FRW3 region.

The NI-1201-F antibody listed in Table 4 comprises the amino acid sequence QQSNSYPLT (SEQ ID NO: 26) in the light chain CDR3 region, the amino acid sequence GIIPAFDTTKYAQKFQG (SEQ ID NO: 42) in the heavy chain CDR2 region, the amino acid sequence DRDILTDYYPLGGMDV (SEQ ID NO: 39) in the heavy chain CDR3 region, and the amino acid sequence TAVYYCAR (SEQ ID NO: 38) in the FRW3 region.

The NI-1201-G antibody listed in Table 4 comprises the amino acid sequence QQSQSYPLT (SEQ ID NO: 41) in the light chain CDR3 region, the amino acid sequence GIIPAFDTTKYAQKFQG (SEQ ID NO: 42) in the heavy chain CDR2 region, the amino acid sequence DRDILTDYYPLGGMDV (SEQ ID NO: 39) in the heavy chain CDR3 region, and the amino acid sequence TAVYYCAR (SEQ ID NO: 38) in the FRW3 region.

TABLE 4
NI-1201 Leads

| | Light | Heavy | Heavy | |
|---|---|---|---|---|
| | Chain | Chain | Chain | |
| | CDR3 | CDR2 | CDR3 | FRW 3 |
| NI1201- | QQSNSYPLT | GIIPLFDTT | DRDILTDY | TAVFYCAR |
| WT | (SEQ ID | KYAQQFQG | YPMGGMDV | (SEQ ID |
| | NO: 26) | (SEQ ID | (SEQ ID | NO: 36) |
| | | NO: 16) | NO: 35) | |
| NI- | QQSNSYPLT | GIIPLFDTT | DRDILTDY | TAVYYCAR |
| 1201-A | (SEQ ID | KYAQKFQG | YPMGGMDV | (SEQ ID |
| | NO: 26) | (SEQ ID | (SEQ ID | NO: 38) |
| | | NO: 37) | NO: 35) | |

### Diseases

The antibodies, formulations, compositions, kits and methods of the disclosure can be useful, without limitation, for the treatment of inflammatory diseases, autoimmune diseases, pulmonary diseases, and oncological diseases.

### Inflammatory Diseases

The disclosure provides compositions, formulations, kits, and methods that can be used in the treatment of inflammatory diseases. In some embodiments, the inflammatory disease is Type II diabetes. In some embodiments, the inflammatory disease is non-alcoholic fatty liver disease (NAFLD). In some embodiments, the inflammatory disease is non-alcoholic steatohepatitis (NASH).

Interleukin-6 (IL-6) is a proinflammatory cytokine that decisively induces the development of insulin resistance and pathogenesis of T2DM through the generation of inflammation by controlling differentiation, migration, proliferation, and cell apoptosis. The presence of IL-6 in tissues is a normal consequence, but its irregular production and long-term exposure leads to the development of inflammation, which induces insulin resistance and overt T2DM. There is a mechanistic relationship between the stimulation of IL-6 and insulin resistance. IL-6 causes insulin resistance by impairing the phosphorylation of insulin receptor and insulin receptor substrate-1 by inducing the expression of SOCS-3, a potential inhibitor of insulin signaling. Thus, antibodies that recognize IL-6, IL-6R and IL-6/IL-6R/gp130 complex and inhibit IL-6 mediated pro-inflammatory signaling may be effective for prophylactic and therapeutic intervention of TlD and T2DM. Accordingly, there exists a need for therapies that neutralize the biological activities CD3 to treat and prevent autoimmune diseases such as diabetes, non-alcoholic steatohepatitis (NASH).

Rheumatoid arthritis (RA) is a chronic, inflammatory disease characterized by progressive, symmetric joint inflammation and subsequent destruction. If untreated, RA is associated with significant patient morbidity and accelerated mortality. Treatment with traditional disease modifying anti-rheumatic drugs (DMARDs) such as methotrexate (MTX) can be efficacious for RA patients. However, appreciation of the severity of the disease and severe side effects make treatment with DMARDs not entirely satisfying. Thus, therapeutic agents targeting pro-inflammatory cytokines that exhibit key roles in the activation and continuation of the destructive process occurring in the rheumatoid synovium. To date, the most notable clinical success in the treatment of RA has been achieved through inhibition of tumor necrosis factor alpha (TNFα) and IL-6.

### Autoimmune Diseases.

The disclosure provides compositions, formulations, kits, and methods that can be used in the treatment of autoimmune diseases. In some embodiments, the autoimmune disease is rheumatoid arthritis. In some embodiments, the autoimmune disease is diabetes. In some embodiments, the diabetes is Type I diabetes. In some embodiments, the autoimmune disease is multiple sclerosis. In some embodiments, the autoimmune disease is lupus (e.g., systemic lupus erythematosus).

Type 1 diabetes (T1D) is caused by the autoimmune destruction of insulin-producing beta cells in the islets of Langerhans, which leads to dependence on exogenous insulin for survival. Despite improvements in care, the desired glycemic targets are not achieved in most patients with type 1 diabetes, and an increased risk of complications and death persists. In genetically susceptible persons, type 1 diabetes progresses through asymptomatic stages before the development of overt hyperglycemia. These stages are characterized by the appearance of autoantibodies (stage 1) and then dysglycemia (stage 2). In type 2 diabetes, metabolic responses to a glucose load are impaired and insulin treatment is not needed. Several immune interventions, when studied in patients with recent-onset clinical type 1 diabetes, have been reported to delay the decline in beta-cell function. One promising type of therapy appears to be Fc receptor-nonbinding anti-CD3 monoclonal antibodies.

The destruction of insulin producing beta cells is primarily by autoimmune disorder which can be corrected by anti-CD3 antibodies. Anti-CD3 antibodies, especially Foralumab may be used by subcutaneous administration to delay the onset and potentially cure diabetes (type 1 and 2). Particularly, this invention is directed to subcutaneous administration of Foralumab either alone or in combination with other anti-inflammatory drugs e.g., anti-IL-6 receptor mAbs to delay the onset of diabetes in juvenile patients.

Multiple sclerosis (MS) is a demyelinating disease of the central nervous system which can affect movement, mobility, gait and vision. MS generally progresses in a relapsing-remitting fashion and treatment is focused on immunosuppression. MS is believed to be driven by T cells, and abnormal expression levels of CD3, CD4, and CD8 on circulating T cells have been described in MS patients. This invention includes the administration of an anti-CD3 antibody and/or an anti-IL-6Rc antibody to treat patients with multiple sclerosis (e.g., to slow progression and improve remission after relapse).

Lupus is an autoimmune disease that causes inflammation in various areas of the body, including the joints, skin, kidney, blood cells, brain, heart and lung. The most common version of lupus is Systemic Lupus Erythematosus (SLE). Increased IL6 levels have been described in SLE patients. This invention includes the administration of an anti-CD3 antibody and/or an anti-IL-6Rc antibody to treat patients with lupus (e.g., to decrease inflammation and inflammatory symptoms).

### Neurodegenerative Diseases

The disclosure provides compositions, formulations, kits, and methods that can be used in the treatment of neurodegenerative diseases. In some embodiments, the neurodegenerative disease is Parkinson's Disease. In some embodiments, the neurodegenerative disease is Alzheimer's Disease.

Parkinson's Disease is a progressive brain disorder which leads to difficulties with walking and coordination, but can also lead to behavioral problems, memory difficulties, and fatigue. Parkinson's Disease is believed to be caused by the loss of dopaminergic neurons leasing to loss of activity of the basal ganglia. Increased levels of IL-6 in the brain have been suggested to underly some of the pathology of Parkinson's Disease. This invention includes the administration of an anti-CD3 antibody and/or an anti-IL-6Rc antibody to treat patients with Parkinson's Disease (e.g., to slow disease progression).

Alzheimer's Disease is a progressive form of dementia affecting predominantly people over the age of 65, although early-onset versions of Alzheimer's do occur. Alzheimer's is believed to be driven by the build-up of amyloid plaques and neurofibrillary, or tau, tangles in the brain. Aberrant IL-6 signaling has been suggested to play a role in the development and progression of Alzheimer's Disease. This invention includes the administration of an anti-CD3 antibody and/or an anti-IL-6Rc antibody to treat patients with Alzheimer's Disease (e.g., to slow disease progression).

### Pulmonary Diseases

The disclosure provides compositions, formulations, kits, and methods that can be used in the treatment of pulmonary diseases. In some embodiments, the pulmonary disease is a pulmonary inflammatory disease. In some embodiments, the pulmonary inflammatory disease is acute respiratory distress syndrome (ARDS). In some embodiment, the pulmonary inflammatory disease is systemic pulmonary sclerosis. In some embodiments, the pulmonary inflammatory disease idiopathic pulmonary fibrosis (IPF),

In some embodiments, a subject treated in accordance with the methods described herein has a disease or pathology associated with coronavirus infection. In some embodiments, the subject has a disease of pathology associated with MERS and/or its variants. In some embodiments, the subject has a disease or pathology associated with SARS and/or its variants.

In some embodiments, the pulmonary disease presents as cytokine release syndrome (CRS).

The disclosure provides methods of treating, preventing, or alleviating a symptom of a pulmonary disease in a subject in need thereof comprising administering to the subject a composition comprising an IL-6R antibody. In some embodiments, the treatment of a pulmonary diseases comprises administering the anti-IL-6Rc antibody to the subject twice, once by subcutaneous injection and once by inhalation. The subcutaneous injection and the inhalation may occur in any order.

### Methods of Treatment

The disclosure provides methods of treating, preventing, or alleviating symptoms of an inflammatory disease, an autoimmune disease, a neurodegenerative disease, a pulmonary disease, or an oncological disease in a subject in need thereof comprising subcutaneously administering to the subject a composition comprising an anti-CD3 antibody, an anti-IL-6Rc antibody, or both.

In some embodiments, the antibody is a monoclonal antibody. In some embodiments, the antibody is fully human or humanized.

In some embodiments, the method of treatment comprises administering an anti-CD3 antibody to a subject in need thereof. In some embodiments, the anti-CD3 antibody comprises a heavy chain complementarity determining region 1 (CDRH1) comprising the amino acid sequence GYGMH (SEQ ID NO: 42), a heavy chain complementarity determining region 2 (CDRH2) comprising the amino acid sequence VIWYDGSKKYYVDSVKG (SEQ ID NO: 43), a heavy chain complementarity determining region 3 (CDRH3) comprising the amino acid sequence QMGYWHFDL (SEQ ID NO: 44), a light chain complementarity determining region 1 (CDRL1) comprising the amino acid sequence RASQSVSSYLA (SEQ ID NO: 45), a light chain complementarity determining region 2 (CDRL2) comprising the amino acid sequence DASNRAT (SEQ ID NO: 46), and a light chain complementarity determining region 3 (CDRL3) comprising the amino acid sequence QQRSNWPPLT (SEQ ID NO: 47).

In some embodiments, the anti-CD3 antibody comprises a variable heavy chain amino acid sequence comprising the amino acid sequence of SEQ ID NO: 48 and a variable light chain amino acid sequence comprising the amino acid sequence of SEQ ID NO: 49.

In some embodiments, the anti-CD3 antibody comprises a heavy chain amino acid sequence comprising the amino acid sequence of SEQ ID NO: 50 and a light chain amino acid sequence comprising the amino acid sequence of SEQ ID NO: 51.

In some embodiments, the autoimmune disease is diabetes or rheumatoid arthritis. In some embodiments, the diabetes is Type 1 diabetes. In some embodiments, the inflammatory disease is Type II diabetes, non-alcoholic fatty liver disease (NAFLD) or non-alcoholic steatohepatitis (NASH). In some embodiments, the oncological disease is hematological cancer. In some embodiments, the hematological cancers is multiple myeloma.

In some embodiments, the anti-CD3 is administered at a daily dose of 1 mg to 5 mg. In some embodiments, the daily dose is administered once daily. In some embodiments, the daily dose is administered for at least 5 consecutive days. In some embodiments, e.g., for the treatment of diabetes, the anti-CD3 antibody is administered daily for up to 2 weeks, up to 3 weeks, up to 4 weeks, up to 5 weeks, or up to 6 weeks. In some embodiments, the anti-CD3 antibody is administered three times a week for two weeks. The administration of three times a week for two weeks may be followed by a week of rest. In some embodiments, e.g., for the treatment of MS, the anti-CD3 antibody is administered three times a week for two weeks, followed by a week of rest for up 12 months.

In some embodiments, the methods comprise administering an antibody that recognizes the Interleukin-6 (IL-6) and IL-6 receptor (IL-6R) complex (IL-6Rc). In some embodiments, the IL-6 Rc antibody comprising a VH CDR1 region comprising the amino acid sequence of SEQ ID NO: 15, a VH CDR2 region comprising the amino acid sequence of SEQ ID NO: 33, a VH CDR3 region comprising the amino acid sequence of SEQ ID NO: 36, a VL CDR1 region comprising the amino acid sequence of SEQ ID NO: 24, a VL CDR2 region comprising the amino acid sequence of SEQ ID NO: 25, and a VL CDR3 region comprising the amino acid sequence of SEQ ID NO: 26. In some embodiments, the IL-6R antibody is Actemra® or Kevzera®. In some embodiments, the antibody is a monoclonal antibody. In some embodiments, the antibody is fully human.

Dosages of IL-6R antibodies administered by nebulizer or inhaler can range from about 10 mg to about 100 mg. In some embodiments, the dose is about 20 mg to about 100 mg. In some embodiments, the dose is about 30 mg to about 100 mg. In some embodiments, the dose is about 40 mg to about 100 mg. In some embodiments, the dose is about 50 mg to about 100 mg. In some embodiments, the dose is about 60 mg to about 100 mg. In some embodiments, the dose is about 70 mg to about 100 mg. In some embodiments, the dose is about 80 mg to about 100 mg. In some embodiments, the dose is about 90 mg to about 100 mg. In some embodiments, the dose is about 25 to about 75 mg. In some embodiments, the dose is about 25 mg. In some embodiments, the dose is about 50 mg. In some embodiments, the dose is about 75 mg.

In some embodiments, the anti-IL-6Rc is administered subcutaneously at a daily dose of 50 mg to 300 mg. In some embodiments, the daily dose is administered once daily. In some embodiments, the daily dose is administered once every 14 days. In some embodiments, the anti-IL-6Rc is administered subcutaneously.

In some embodiments, a method provided herein comprises administering to a subject both an anti-CD3 antibody and an anti-IL6Rc antibody. The anti-IL-6Rc antibody may be administered prior to, concurrently with, or subsequently to the anti-CD3 antibody. The anti-CD3 antibody and the anti-IL6Rc antibody may individually be administered subcutaneously, intranasally, or by inhalation.

In some embodiments, a method provided herein comprises administering an anti-CD3 antibody described herein by subcutaneous injection followed by administration of an anti-IL-6Rc antibody described herein by subcutaneous injection. In some embodiments, a method provided herein comprises administering an anti-IL6Rc antibody described herein by subcutaneous injection followed by administration of an anti-CD3 antibody described herein by subcutaneous injection.

In some embodiments, a method provided herein comprises two administrations of an anti-CD3 antibody by two different routes of administration. For example, in some embodiments, a method provided herein comprises a first subcutaneous administration of an anti-CD3 antibody described herein followed by a second administration of an anti-CD3 antibody by inhalation. In some embodiments, a method provided herein comprises a first administration of an anti-CD3 antibody described herein by inhalation followed by a second subcutaneous administration of an anti-CD3 antibody. In some embodiments, a method provided herein comprises a first subcutaneous administration of an anti-CD3 antibody described herein followed by a second intranasal administration of an anti-CD3 antibody. In some embodiments, a method provided herein comprises a first intranasal administration of an anti-CD3 antibody described herein followed by a second subcutaneous administration of an anti-CD3 antibody.

In some embodiments, a method provided herein comprises two administrations of an anti-IL6Rc antibody by two different routes of administration. For example, in some embodiments, a method provided herein comprises a first subcutaneous administration of an anti-IL6Rc antibody described herein followed by a second administration of an anti-IL6Rc antibody by inhalation. In some embodiments, a method provided herein comprises a first administration of an anti-IL6Rc antibody described herein by inhalation followed by a second subcutaneous administration of an anti-IL6Rc antibody. In some embodiments, a method provided herein comprises a first subcutaneous administration of an anti-IL6Rc antibody described herein followed by a second intranasal administration of an anti-IL6Rc antibody. In some embodiments, a method provided herein comprises a first intranasal administration of an anti-IL6Rc antibody described herein followed by a second subcutaneous administration of an anti-IL6Rc antibody.

In some embodiments, a method provided herein comprises two subsequence subcutaneous administrations of an anti-CD3 antibody or an anti-IL6Rc antibody. The two subcutaneous administrations may occur at the same site or at different sites of the body.

In one embodiment, antibodies of the invention, which include a monoclonal antibody of the invention (e.g., a fully human monoclonal antibody), may be used as therapeutic agents. Such agents will generally be employed to treat, alleviate, and/or prevent a disease or pathology associated with an inflammatory, autoimmune or oncological disease in a subject. A therapeutic regimen is carried out by identifying a subject, e.g., a human patient suffering from (or at risk of developing) with an inflammatory, autoimmune or oncological disease, using standard methods. An antibody preparation, preferably one having high specificity and high affinity for its target antigen, is administered to the subject and will generally have an effect due to its binding with the target.

In some embodiments, the subject is a human subject.

A therapeutically effective amount of an antibody of the invention relates generally to the amount needed to achieve a therapeutic objective. As noted above, this may be a binding interaction between the antibody and its target antigen that, in certain cases, interferes with the functioning of the target. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Efficaciousness of treatment is determined in association with any known method for diagnosing or treating an inflammatory, autoimmune or oncological disease. Alleviation of one or more symptoms of with an inflammatory, autoimmune or oncological disease indicates that the antibody confers a clinical benefit.

The methods of treatment or prevention typically include administering subcutaneously to a subject an anti-CD-3 antibody composition sufficient to stimulate the immune system. In some embodiments, the methods include administering an anti-CD3 antibody composition sufficient to increase IL-10 and/or TGF-.beta. production by T cells in the peripheral blood, e.g., regulatory T cells, e.g., by about 100%, 200%, 300% or more. In some embodiments, the methods include administering an anti-CD3 antibody composition sufficient to decrease T cell proliferation in the peripheral blood, e.g., by about 20%; e.g., in some embodiments, by at least about 20%, 30%, 40%, 50%, 60%, 70%, 80%, 90%, or more.

In some embodiments, the methods can include administering to the subject an anti-IL6Rc antibody, before, concomitantly with, or after administration of the anti-CD3 compositions.

The methods provided herein may further comprise the administration of an additional therapeutic agent. In some embodiments, the additional therapeutic agent is indicated for the treatment of the disease which is being treated by administration of the anti-CD3 antibody and/or anti-IL-6Rc antibody. In some embodiments, the additional therapeutic agent is an antibody. In some embodiments, the additional; therapeutic agent is an IL-6 antagonist.

### Formulations, Compositions, and Kits.

The anti-CD3 and the anti-IL-6Rc antibodies described herein can be incorporated into a pharmaceutical composition suitable for subcutaneous, intranasal, or intravenous administration or for administration by inhalation.

In some embodiments, the composition comprises an anti-CD3 antibody comprising a heavy chain complementarity determining region 1 (CDRH1) comprising the amino acid sequence GYGMH (SEQ ID NO: 42), a heavy chain complementarity determining region 2 (CDRH2) comprising the amino acid sequence VIWYDGSKKYYVDSVKG (SEQ ID NO: 43), a heavy chain complementarity determining region 3 (CDRH3) comprising the amino acid sequence QMGYWHFDL (SEQ ID NO: 44), a light chain complementarity determining region 1 (CDRL1) comprising the amino acid sequence RASQSVSSYLA (SEQ ID NO: 45), a light chain complementarity determining region 2 (CDRL2) comprising the amino acid sequence DASNRAT (SEQ ID NO: 46), and a light chain complementarity determining region 3 (CDRL3) comprising the amino acid sequence QQRSNWPPLT (SEQ ID NO: 47).

In some embodiments, the anti-CD3 antibody comprises a variable heavy chain amino acid sequence comprising the amino acid sequence of SEQ ID NO: 48 and a variable light chain amino acid sequence comprising the amino acid sequence of SEQ ID NO: 49.

In some embodiments, the anti-CD3 antibody comprises a heavy chain amino acid sequence comprising the amino acid sequence of SEQ ID NO: 50 and a light chain amino acid sequence comprising the amino acid sequence of SEQ ID NO: 51.

In some embodiments, the composition comprises an antibody that recognizes the Interleukin-6 (IL-6) and IL-6 receptor (IL-6R) complex (IL-6Rc). In some embodiments, the anti-antibody comprises the IL-6 R antibody comprising a VH CDR1 region comprising the amino acid sequence of SEQ ID NO: 15, a VH CDR2 region comprising the amino acid sequence of SEQ ID NO: 33, a VH CDR3 region comprising the amino acid sequence of SEQ ID NO: 36, a VL CDR1 region comprising the amino acid sequence of SEQ ID NO: 24, a VL CDR2 region comprising the amino acid sequence of SEQ ID NO: 25, and a VL CDR3 region comprising the amino acid sequence of SEQ ID NO: 26. In some embodiments, the IL-6R antibody is Actemra® or Kevzera®. In some embodiments, the antibody is a monoclonal antibody. In some embodiments, the antibody is fully human.

In some embodiments, the composition comprises IL-6R antibody between about 5 mg/mL and 50 mg/mL. In some embodiments, the composition comprises IL-6R antibody at about 5 mg/mL. In some embodiments, the composition comprises IL-6R antibody at about 10 mg/mL. In some embodiments, the composition comprises IL-6R antibody at about 15 mg/mL. In some embodiments, the composition comprises IL-6R antibody at about 20 mg/mL. In some embodiments, the composition comprises IL-6R antibody at about 25 mg/mL. In some embodiments, the composition comprises IL-6R antibody at about 30 mg/mL. In some embodiments, the composition comprises IL-6R antibody at about 35 mg/mL. In some embodiments, the composition comprises IL-6R antibody at about 40 mg/mL. In some embodiments, the composition comprises IL-6R antibody at about 45 mg/mL. In some embodiments, the composition comprises IL-6R antibody at about 50 mg/mL. In some embodiments, the composition comprises IL-6R antibody at about 100 mg/mL. In some embodiments, the composition comprises IL-6R antibody at about 150 mg/mL. In some embodiments, the composition comprises IL-6R antibody at about 200 mg/mL.

In some embodiments, a composition for subcutaneous administration comprises IL-6R antibody at about 100 mg/mL. In some embodiments, a composition for subcutaneous administration comprises IL-6R antibody at up to about 150 mg/mL. In some embodiments, a composition for subcutaneous administration comprises IL-6R antibody at about 200 mg/mL.

In some embodiments, a composition for administration by inhalation comprises IL-6R antibody at about 10 mg/mL. In some embodiments, a composition for administration by inhalation comprises IL-6R antibody at up to about 15 mg/mL. In some embodiments, a composition for administration by inhalation comprises IL-6R antibody at about 20 mg/mL. In some embodiments, a composition for administration by inhalation comprises IL-6R antibody at about 25 mg/mL.

Principles and considerations involved in preparing such compositions, as well as guidance in the choice of components are provided, for example, in Remington's Pharmaceutical Sciences: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

Such compositions typically comprise the antibody and a pharmaceutically acceptable carrier. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. (See, e.g., Marasco et al., Proc. Natl. Acad. Sci. USA, 90: 7889-7893 (1993)).

As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated.

The formulations to be used for in vivo administration must be sterile. This is readily accomplished by filtration through sterile filtration membranes.

A pharmaceutical composition of the invention is formulated to be compatible with its intended route of administration. Solutions or suspensions used for subcutaneous or intravenous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The reparation can be enclosed in ampoules, disposable syringes or multiple dose vials made of glass or plastic.

The active compound is administered by nasal inhalation, inhalation through the mouth, intravenously, orally, or any combination thereof. Alternatively the, active compound is administered orally via an enteric-coated capsule.

Dosage, toxicity and therapeutic efficacy of such antibody compositions can be determined by standard pharmaceutical procedures in cell cultures (e.g., of cells taken from an animal after simultaneous or intravenous administration of an antibody) or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions which exhibit high therapeutic indices are preferred. While anti-CD3 or IL-6R antibody compositions that exhibit toxic side effects may be used, care should be taken to design a delivery system that targets such compounds to the site of affected tissue in order to minimize potential damage and, thereby, reduce side effects.

The data obtained from the cell cultures (e.g., of cells taken from an animal after mucosal administration of an anti-CD3 antibody) and animal studies can be used in formulating a range of dosage for use in humans. The dosage of anti-CD3 and or IL-6R antibody compositions lies preferably within a range of circulating concentrations that include the ED₅₀ with little or no toxicity. The dosage may vary within this range depending upon the dosage form employed and the route of administration utilized. For antibody compositions used in the methods described herein, the therapeutically effective dose can be estimated initially from assays of cell cultures A dose may be formulated in animal models to achieve a desired circulating plasma concentration of IL-10 or TGFβ, or of regulatory cells, in the range that includes the IC₅₀ (i.e., the concentration of the test compound which achieves a half-maximal inhibition of symptoms) as determined in cell culture. Such information can be used to more accurately determine useful doses in humans. Levels of IL-10 or TGFβ. in plasma can be measured by methods known in the art, for example, by ELISA. Levels of regulatory cells can be measured by methods known in the art, for example, by flow cytometry-based methods.

As defined herein, a therapeutically effective amount of an anti-CD3 or anti-IL-6R antibody (i.e., an effective dosage) depends on the antibody selected, the mode of delivery, and the condition to be treated. For instance, single dose amounts in the range of approximately 1:g/kg to 1000 g/kg may be administered; in some embodiments, about 5, 10, 50, 100, or 500:g/kg may be administered. In some embodiments, e.g., pediatric subjects, about 1 to 100:g/kg, e.g., about 25 or 50:g/kg, of anti-CD3 or anti-IL-6R antibody can be administered. The anti-CD3 and/or anti-IL-6R antibody compositions can be administered from one or more times per day to one or more times per week; including once every other day. The subcutaneous anti-CD3 antibody compositions can be administered, e.g., for about 10 to 14 days or longer. For example, the CD3 antibody can be administered once a day for 5 or more days and the e IL6-Rc antibody can be administered once every 14 days. This cycle or administration can be repeated as necessary to treat, prevent or alleviate a symptom of disease. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds can include a single treatment or, can include a series of treatments.

The pharmaceutical compositions can be included in a container, pack, or dispenser together with instructions for administration.

### Injectable Formulations

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Formulations and compositions appropriate for administration by subcutaneous injection can include hyaluronidsase enzymes.

“Hyaluronan” (abbreviated “HA” and also called “hyaluronic acid” or “hyaluronate”) is an anionic, nonsulfated glycosaminoglycan distributed widely throughout connective, epithelial, and neural tissues.

“Hyaluronidases” are enzymes that degrade hyaluronic acid. In humans, there are six associated genes, including HYALPI (pseudogene), HYAL1, HYAL2, HYAL3, HYAL4, and PH20/SPAM1. The term herein includes “acid-active” enzymes (such as HYAL1), and “neutral-active” enzymes (such as PH20). It also includes enzymes with or without a glycosylphosphtidy inositol anchor; preferably the hyaluronidase is soluble or lacks an anchor. Hyaluronidases can be included in a pharmaceutical formulation in order to: facilitate administration of the therapeutic drug into the hypodermis, reduce the viscosity of the interstitum, allow larger volumes to be administered SC, and/or increase absorption and dispersion of another injected drug. The hyaluronidase enzyme in a pharmaceutical formulation herein is characterized by having no adverse effect on the molecular integrity of the anti-IL-6R antibody in the formulation, and while it modifies the delivery of the anti-IL-6R antibody to the systemic circulation it does not possess any properties that could provide or contribute to the therapeutic effects of systemically absorbed anti-IL-6R antibody. See, also, WO 2004/078140, WO2006/091871 and U.S. Pat. No. 7,767,429 regarding hyaluronidases according to the present invention. Hyaluronidase products approved in EU countries include HYALASE@. Hyaluronidase products of animal origin approved in the US include VITRASE™, HYDRASE™ and AMPHADASE™. The preferred hyaluronidase herein is recombinant human PH20.

“Recombinant human PH20” (abbreviated “rHuPH20”) refers to a soluble, neutral pH-active enzyme comprising a truncated human PH20 amino acid sequence. It can be synthesized with a 35 amino acid signal peptide that is removed from the N-terminus during the process of secretion so as to provide an N-terminal amino acid sequence found in some bovine hyaluronidase preparations. Preferably, rHuPH20 herein comprises the amino acid sequence available under CAS Registry No. 757971-58-7 or as disclosed in U.S. Pat. No. 7,767,429, expressly incorporated herein by reference, and has an approximate molecular weight of 61 kDa. See, also, Frost, G. I., “Recombinant human hyaluronidase (rHuPH20): an enabling platform for subcutaneous drug and fluid administration”, Expert Opinion on Drug Delivery 4: 427-440 (2007)). The term herein includes rHuPH20 (HYLENEX®) commercially available from Halozyme Therapeutics Inc..

Preferred subcutaneous dose ranges for an anti-CD3 antibody described herein are between 0.1 mg to 5 mg daily. For example, a dose of about 0.1 mg, about 0.2 mg, about 0.3 mg, about 0.4 mg, about 0.5 mg, about 0.6 mg, about 0.7 mg, about 0.8 mg, about 0.9 mg, about 1.0 mg, about 1.5 mg, about 2.0 mg, about 2.5 mg, about 3.0 mg, about 3.5 mg, about 4.0 mg, or about 5.0 mg is administered daily. In some embodiments, a dose of about 0.1 mg to about 2 mg is administered daily. Administration of the dose is once daily or twice daily.

Preferred subcutaneous dose ranges for anti-IL-6Rc antibody described herein are between 50 mg to 300 mg daily. For example, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, or about 300 mg may be administered daily.

A preferred formulation for subcutaneous administration is a preferred dosage of anti-CD3 antibody in 25 mM sodium acetate buffer, 125 mM sodium chloride with 0.02% polysorbate 80, at pH 5.5.

Suitable injection volume for subcutaneous administration range from about 0.1 mL to 2 mL. In some embodiments, about 0.1-0.3 mL, about 0.3-0.5 mL, about 0.5-0.7 mL, about 0.7-1.1 mL, about 1.1-1.3 mL, about 1.3-1.5 mL, about 1.5-1.7 mL, or about 1.7-1.9 mL of a formulation are administered subcutaneously. In some embodiments, no more than 2 mL of formulation are administered subcutaneously.

The pharmaceutical compositions and subcutaneous or intravenous dosage forms can further comprise one or more compounds that reduce the rate by which an active ingredient will decompose.

Such compounds, which are referred to herein as “stabilizers,” include, but are not limited to, antioxidants such as ascorbic acid and salt buffers. For example, cellulose acetate phthalate, polyvinyl acetate phthalate, hydroxypropylmethyl cellulose phthalate, methacrylic acid-methacrylic acid ester copolymers, cellulose acetate trimellitate, carboxymethylethyl cellulose, and hydroxypropylmethyl cellulose acetate succinate, among others, can be used to achieve enteric coating. Mixtures of waxes, shellac, zein, ethyl cellulose, acrylic resins, cellulose acetate, silicone elastomers can be used to achieve sustained release coating. See, for example, Remington, supra, Chapter 93, for other types of coatings, techniques and equipment.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In one embodiment, the subcutaneous or intravenous anti-CD3 or anti-IL-6Rc antibody compositions are prepared with carriers that will protect the antibody against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

### Formulations for Inhalation

For administration by inhalation, the compounds are delivered in the form of an aerosol spray from pressured container or dispenser which contains a suitable propellant, e.g., a gas such as carbon dioxide, or a nebulizer.

Administration by inhalation may be in the form of an inhaler or a nebulizer. The nebulizer and/or inhaler is handheld. Optionally, the nebulizer and/or inhaler can be of different sizes to fit children and/or adults.

The active compound may be formulated as a particle and keep the drug particle at a desired range of particle sizes. The particle size range is, for example, between 2-10 microns. Particles of a particle formulation have diameters of between about 1 mm to about 5 mm, e.g., less than 5 mm in diameter, less than 4 mm in diameter, less than 3 mm in diameter, less than 2 mm in diameter, and about 1 mm in diameter.

Particles of a particle formulation comprising an anti-CD3 antibody or antigen-binding fragment thereof have average diameters of between about 0.1 mm to about 50 mm. Particles of a particle formulation comprising an anti-CD3 antibody or antigen-binding fragment thereof have average diameters of between about 1 mm to about 10 mm, e.g., less than 10 mm in average diameter, less than 9 mm in average diameter, less than 8 mm in average diameter, less than 7 mm in average diameter, less than 6 mm in average diameter, less than 5 mm in average diameter, less than 4 mm in average diameter, less than 3 mm in average diameter, and about 2 mm in average diameter. In some embodiments, particles have average diameters of between about 2 mm and 5 mm. In some embodiments, the particles have an average diameter between 2 mm and 5 mm, where each particle is less than about 50 mm in diameter.

In some embodiments the formulation is an extended and controlled release formulation. Methods of producing extended and controlled release formulation are known in the art and includes for example the use or macroporous beads.

In some embodiments, a formulation, or composition, of the disclosure contains excipients such as stabilizers, preservative, phospholipids and/or other ingredients to improve stability and shelf life and in the case of particles a uniform particle size. In some embodiments, the formulation or composition is a pharmaceutically acceptable composition. Exemplary excipients include, but are not limited to surfactants such as Trehalose (1-20%), emulsifiers such as polysorbate 20 (0.01%-0.1%) or polysorbate 80 (0.01%-0.1%), sodium chloride (50-200 mM), EDTA or EGTA (0.1 to 1 mM), a buffer such as histidine buffer (1-50 mM) or sodium citrate buffer (10-50 mM). Acceptable pH ranges for formulations can be, for example, 4.0 to 7.0.

Preferred inhalation dose ranges for an anti-CD3 antibody are between 0.1 mg to 5 mg daily. For example, a dose of about 0.1 mg, about 0.2 mg, about 0.3 mg, about 0.4 mg, about 0.5 mg, about 0.6 mg, about 0.7 mg, about 0.8 mg, about 0.9 mg, about 1.0 mg, about 1.5 mg, about 2.0 mg, about 2.5 mg, about 3.0 mg, about 3.5 mg, about 4.0 mg, or about 5.0 mg is administered daily. Administration of the dose is once daily or twice daily.

Dosages of IL-6Rc antibodies administered by nebulizer or inhaler can range from about 10 mg to about 100 mg. In some embodiments, the dose is about 20 mg to about 100 mg. In some embodiments, the dose is about 30 mg to about 100 mg. In some embodiments, the dose is about 40 mg to about 100 mg. In some embodiments, the dose is about 50 mg to about 100 mg. In some embodiments, the dose is about 60 mg to about 100 mg. In some embodiments, the dose is about 70 mg to about 100 mg. In some embodiments, the dose is about 80 mg to about 100 mg. In some embodiments, the dose is about 90 mg to about 100 mg. In some embodiments, the dose is about 25 to about 75 mg. In some embodiments, the dose is about 25 mg. In some embodiments, the dose is about 50 mg. In some embodiments, the dose is about 75 mg.

The volume administered by nebulizer may range from about 0.5 mL to about 5 mL. In some embodiments, about 0.5 mL, about 1 mL, about 1.5 mL, about 2 mL, about 2.5 mL, about 3 mL, about 3.5 mL, about 4 mL, about 4.5 mL or about 5 mL are administered by nebulizer.

In some embodiments, the composition comprises an IL-6Rc antibody, histidine, sodium chloride, and polysorbate 80. In some embodiments, the composition comprises an IL-6Rc antibody, histidine, sodium chloride, and polysorbate 20. In some embodiments, the histidine is about 5 mM, about 10 mM, about 15 mM, about 20 mM, about 25 mM, about 30 mM, about 35 mM, or about 40 mM. In some embodiments, the histidine is about 25 mM. In some embodiments, the sodium chloride is about 50 mM, about 75 mM, about 100 mM, about 125 mM, about 150 mM, about 175 mM or about 200 mM. In some embodiments, the sodium chloride is about 125 mM. In some embodiments, the polysorbate 80 is about 0.01%, about 0.02%, about 0.03%, about 0.04%, about 0.05%, about 0.06%, about 0.07%, about 0.08%, about 0.09%, or about 0.1%. In some embodiments, the polysorbate 80 is about 0.02%. In some embodiments, the polysorbate 80 is about 0.05%. In some embodiments, the composition has a pH of about 5.0, about 6.0, or about 7.0. In some embodiments, the composition has a pH of about 6.0. In some embodiments, the IL-6Rc antibody is about 5 mg/mL, about 10 mg/mL, about 15 mg/mL, about 20 mg/mL, about 25 mg/mL, about 30 mg/mL, or about 35 mg/mL. In some embodiments, the IL-6Rc antibody is about 20 mg/mL. In some embodiments, the histidine is 25 mM, the sodium chloride is 125 mM, the polysorbate 80 is 0.02%, the pH is 6.0, and the IL-6Rc antibody is 20 mg/mL. In some embodiments, the histidine is 25 mM, the sodium chloride is 125 mM, the polysorbate 80 is 0.05%, the pH is 6.0, and the IL-6Rc antibody is 20 mg/mL.

In some embodiments, a vial containing a stabilized and formulated solution or composition of anti-IL-6 receptor (anti-IL-6R) mAb and combinations as described herein is inserted into an inhaler and/or nebulizer. In some embodiments, a vial containing a stabilized and formulated solution of anti-IL-6 receptor (anti-IL-6R) mAb and combinations as described herein is inserted into the bottom of the inhaler and/or nebulizer. In some embodiments, the drug solution is dispensed as fine aerosols through the mouth.

### Formulations for Intranasal Administration

For nasal administration, the formulations may be an aerosol in a sealed vial or other suitable container. Preferred nasal dose ranges for an anti-CD3 antibody provided herein are between 0.01 mg to 1 mg daily. For example, a dose of about 0.01 mg, about 0.05 mg, about 0.06 mg, about 0.07 mg, about 0.08 mg, about 0.09 mg, about 0.1 mg, about 0.2 mg, about 0.25 mg about 0.3 mg, about 0.4 mg, about 0.5 mg, about 0.6 mg, about 0.7 mg, about 0.8 mg, about 0.9 mg, or about 1.0 mg, may be administered daily. In some embodiments, about 10 μg to 250 μg is administered daily. The does is equally split between each nostril. Administration of the dose is once daily or twice daily.

In some embodiments, a formulation, or composition, of the disclosure contains excipients such as stabilizers, preservative, phospholipids and/or other ingredients to improve stability and shelf life and in the case of particles a uniform particle size. In some embodiments, the formulation or composition is a pharmaceutically acceptable composition. Exemplary excipients include, but are not limited to surfactants such as Trehalose (1-20%), emulsifiers such as polysorbate 20 (0.01%-0.1%) or polysorbate 80 (0.01%-0.1%), sodium chloride (50-200 mM), EDTA or EGTA (0.1 to 1 mM), a buffer such as histidine buffer (1-50 mM) or sodium citrate buffer (10-50 mM). Acceptable pH ranges for formulations can be, for example, 4.0 to 7.0.

In some embodiments, the nasal antibody formulation comprises a population of particles having a particle size in the range of about 1 mm to about 5 mm.

In some embodiments, the nasal antibody formulation includes a solution comprising an antibody at a dosage in the range of about 0.1 mg to about 10 mg, a sodium citrate buffer at a concentration in the range of about 25 mm to about 50 mm, and sodium chloride at a concentration of about 150 mm, where the solution has a pH in the range of about 4 to 6.

In some embodiments, the nasal antibody formulation includes one or more polyols as stabilizing excipients. In some embodiments, the polyol is mannitol at a concentration in the range of about 0.10% to about 10%. In some embodiments, the polyol is trehalose at a concentration in the range of about 0.1% to about 1%. In some embodiments, the polyol is sorbitol at a concentration in the range of about 1% to about 10%. In some embodiments, the polyol is glycerol at a concentration in the range of about 1% to about 10%. In some embodiments, the polyol is mannitol at a concentration in the range of about 0.10% to about 10%, and trehalose at a concentration in the range of about 0.1% to about 1%. In some embodiments, the polyol is mannitol at a concentration in the range of about 0.10% to about 10%, and sorbitol at a concentration in the range of about 1% to about 10%. In some embodiments, the nasal antibody formulation includes one or more polyols as stabilizing excipients, and glycerol at a concentration in the range of about 1% to about 10%. In some embodiments, the polyol is trehalose at a concentration in the range of about 0.10% to about 10%, and sorbitol at a concentration in the range of about 1% to about 10%. In some embodiments, the polyol is trehalose at a concentration in the range of about 0.10% to about 1%, and glycerol at a concentration in the range of about 1% to about 10%. In some embodiments, the polyol is sorbitol at a concentration in the range of about 1% to about 10%, and glycerol at a concentration in the range of about 1% to about 10%. In some embodiments, the polyol is mannitol at a concentration in the range of about 0.10% to about 10%, trehalose at a concentration in the range of about 0.10% to about 1%, and sorbitol at a concentration in the range of about 1% to about 10%. In some embodiments, the polyol is mannitol at a concentration in the range of about 0.1% to about 10%, trehalose at a concentration in the range of about 0.10% to about 10%, and glycerol at a concentration in the range of about 10% to about 10%. In some embodiments, the polyol is trehalose at a concentration in the range of about 0.10% to about 1%, sorbitol at a concentration in the range of about 1% to about 10%, and glycerol at a concentration in the range of about 1% to about 10%. In some embodiments, the polyol is mannitol at a concentration in the range of about 0.10% to about 10%, trehalose at a concentration in the range of about 0.10% to about 1%, sorbitol at a concentration in the range of about 1% to about 10%, and the polyol is glycerol at a concentration in the range of about 1% to about 10%.

In some embodiments, the nasal antibody formulation includes one or more surfactants such as, by way of non-limiting example, Polysorbate 20 or Polysorbate 80. In some embodiments, the Polysorbate 20 or Polysorbate 80 is present at a concentration in the range of about 0.01% to about 0.05%.

In some embodiments, the nasal antibody formulation is suitable for storage at about 2° C. to about 4° C. In some embodiments, the nasal anti-CD3 antibody formulation is stored in a sealed vial or other suitable container. In some embodiments, the nasal anti-CD3 antibody formulation is stored in a sealed vial or other suitable container at about 2° C. to about 4° C.

### Kits

The kits described herein can include an anti-CD3 antibody composition and/or anti-IL-6Rc as an already prepared subcutaneous or intravenous dosage form ready for administration or, alternatively, can include an anti-CD3 antibody or anti-IL-6Rc composition as a solid pharmaceutical composition that can be reconstituted with a solvent to provide a liquid dosage form suitable or subcutaneous or intravenous administration. When the kit includes an anti-CD3 or anti-IL-6Rc antibody composition as a solid pharmaceutical composition that can be reconstituted with a solvent to provide a liquid dosage form (e.g., for subcutaneous or intravenous administration), the kit may optionally include a reconstituting solvent. In this case, the constituting or reconstituting solvent is combined with the active ingredient to provide a liquid oral dosage form of the active ingredient. Typically, the active ingredient is soluble in the solvent and forms a solution. The solvent can be, e.g., water, a non-aqueous liquid, or a combination of a non-aqueous component and an aqueous component. Suitable non-aqueous components include, but are not limited to oils; alcohols, such as ethanol; glycerin; and glycols, such as polyethylene glycol and propylene glycol. In some embodiments, the solvent is phosphate buffered saline (PBS).

### Definitions

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As used herein, the term “antibody” refers to immunoglobulin molecules and immunologically active portions of immunoglobulin (Ig) molecules, i.e., molecules that contain an antigen binding site that specifically binds (immunoreacts with) an antigen. By “specifically bind” or “immunoreacts with” “or directed against” is meant that the antibody reacts with one or more antigenic determinants of the desired antigen and does not react with other polypeptides or binds at much lower affinity (K d>10-6). Antibodies include, but are not limited to, polyclonal, monoclonal, chimeric, dAb (domain antibody), single chain, F ab, F ab′ and F (ab′)2 fragments, scFvs, and an Fab expression library

As used in this specification and the appended claims, the singular forms “a,” “an” and “the” include plural references unless the content clearly dictates otherwise.

As used in this specification, the term “and/or” is used in this disclosure to mean either “and” or “or” unless indicated otherwise.

Throughout this specification, unless the context requires otherwise, the words “comprise”, or variations such as “comprises” or “comprising”, will be understood to imply the inclusion of a stated element or integer or group of elements or integers but not the exclusion of any other element or integer or group of elements or integers.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The term “antibody” as used herein refers to an immunoglobulin molecule or immunologically active portion thereof, i.e., an antigen-binding portion. Examples of immunologically active portions of immunoglobulin molecules include F(ab) and F(ab′)2 fragments, which retain the ability to bind a target antigen (e.g. IL-6Rc or CD3). Such fragments can be obtained commercially, or using methods known in the art. For example, F(ab)2 fragments can be generated by treating the antibody with an enzyme such as pepsin, a non-specific endopeptidase that normally produces one F(ab)2 fragment and numerous small peptides of the Fc portion. The resulting F(ab)2 fragment is composed of two disulfide-connected Fab units. The Fc fragment is extensively degraded and can be separated from the F(ab)2 by dialysis, gel filtration or ion exchange chromatography. F(ab) fragments can be generated using papain, a non-specific thiol-endopeptidase that digests IgG molecules, in the presence of a reducing agent, into three fragments of similar size: two Fab fragments and one Fc fragment. When Fc fragments are of interest, papain is the enzyme of choice because it yields a 50,00 Dalton Fc fragment; to isolate the F(ab) fragments, the Fc fragments can be removed, e.g., by affinity purification using protein A/G. A number of kits are available commercially for generating F(ab) fragments, including the ImmunoPure IgGI Fab and F(ab′)2. Preparation Kit (Pierce Biotechnology, Rockford, Ill.). In addition, commercially available services for generating antigen-binding fragments can be used, e.g., Bio Express, West Lebanon, N.H.

The term “cytokine” refers to all human cytokines known within the art that bind extracellular receptors expressed on the cell surface and thereby modulate cell function, including but not limited to IL-2, IFN-gamma, TNF-α, IL-4, IL-5, IL-6, IL-9, IL-10, and IL-13.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. In general, antibody molecules obtained from humans relate to any of the classes IgG, IgM, IgA, IgE and IgD, which differ from one another by the nature of the heavy chain present in the molecule. Certain classes have subclasses as well, such as IgG 1, IgG 2, and others. Furthermore, in humans, the light chain may be a kappa chain or a lambda chain.

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

In general, antibody molecules obtained from humans relate to any of the classes IgG, IgM, IgA, IgE and IgD, which differ from one another by the nature of the heavy chain present in the molecule. Certain classes have subclasses as well, such as IgG 1, IgG 2, and others. Furthermore, in humans, the light chain may be a kappa chain or a lambda chain.

The term “antigen-binding site” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987), Chothia et al. Nature 342:878-883 (1989).

As used herein, the term “epitope” includes any protein determinant capable of specific binding to an immunoglobulin or fragment thereof, or a T-cell receptor. The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. An antibody is said to specifically bind an antigen when the dissociation constant is ≤1 μM; e.g., ≤100 nM, preferably ≤10 nM and more preferably ≤1 nM.

As used herein, the terms “immunological binding,” and “immunological binding properties” refer to the non-covalent interactions of the type which occur between an immunoglobulin molecule and an antigen for which the immunoglobulin is specific. The strength, or affinity of immunological binding interactions can be expressed in terms of the dissociation constant (Kd) of the interaction, wherein a smaller Kd represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kon) and the “off rate constant” (Koff) can be determined by calculation of the concentrations and the actual rates of association and dissociation. (See Nature 361:186-87 (1993)). The ratio of Koff/Kon enables the cancellation of all parameters not related to affinity, and is equal to the dissociation constant Kd. (See, generally, Davies et al. (1990) Annual Rev Biochem 59:439-473). An antibody of the present invention is said to specifically bind to IL-6Rc and/or both IL-6Rc and IL-6R, when the equilibrium binding constant (K d) is ≤1 μM, preferably ≤100 nM, more preferably ≤10 nM, and most preferably ≤100 pM to about 1 pM, as measured by assays such as radioligand binding assays or similar assays known to those skilled in the art.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence. Polynucleotides in accordance with the invention include the nucleic acid molecules encoding the heavy chain immunoglobulin molecules presented in SEQ ID NOS: 2, 8 and 12, and nucleic acid molecules encoding the light chain immunoglobulin molecules represented in SEQ ID NOS: 4, 6, 10, and 14.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g., free of marine proteins, (3) is expressed by a cell from a different species, or (4) does not occur in nature.

The term “polypeptide” is used herein as a generic term to refer to native protein, fragments, or analogs of a polypeptide sequence. Hence, native protein fragments, and analogs are species of the polypeptide genus. Polypeptides in accordance with the invention comprise the heavy chain immunoglobulin molecules represented in SEQ ID NOS: 2, 8, and 12, and the light chain immunoglobulin molecules represented in SEQ ID NOS: 4, 6, 10, and 14 as well as antibody molecules formed by combinations comprising the heavy chain immunoglobulin molecules with light chain immunoglobulin molecules, such as kappa light chain immunoglobulin molecules, and vice versa, as well as fragments and analogs thereof.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

The term “control sequence” as used herein refers to polynucleotide sequences which are necessary to effect the expression and processing of coding sequences to which they are ligated. The nature of such control sequences differs depending upon the host organism in prokaryotes, such control sequences generally include promoter, ribosomal binding site, and transcription termination sequence in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences. The term “polynucleotide” as referred to herein means a polymeric boron of nucleotides of at least 10 bases in length, either ribonucleotides or deoxynucleotides or a modified form of either type of nucleotide. The term includes single and double stranded forms of DNA.

The term “oligonucleotide” referred to herein includes naturally occurring, and modified nucleotides linked together by naturally occurring, and non-naturally occurring oligonucleotide linkages. Oligonucleotides are a polynucleotide subset generally comprising a length of 200 bases or fewer. Preferably oligonucleotides are 10 to 60 bases in length and most preferably 12, 13, 14, 15, 16, 17, 18, 19, or 20 to 40 bases in length. Oligonucleotides are usually single stranded, e.g., for probes, although oligonucleotides may be double stranded, e.g., for use in the construction of a gene mutant. Oligonucleotides of the invention are either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes Oligonucleotides linkages such as phosphorothioate, phosphorodithioate, phosphoroselerloate, phosphorodiselenoate, phosphoroanilothioate, phoshoraniladate, phosphoronmidate, and the like. See e.g., LaPlanche et al. Nucl. Acids Res. 14:9081 (1986); Stec et al. J. Am. Chem. Soc. 106:6077 (1984), Stein et al. Nucl. Acids Res. 16:3209 (1988), Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990). An oligonucleotide can include a label for detection, if desired.

The term “selectively hybridize” referred to herein means to detectably and specifically bind. Polynucleotides, oligonucleotides and fragments thereof in accordance with the invention selectively hybridize to nucleic acid strands under hybridization and wash conditions that minimize appreciable amounts of detectable binding to nonspecific nucleic acids. High stringency conditions can be used to achieve selective hybridization conditions as known in the art and discussed herein. Generally, the nucleic acid sequence homology between the polynucleotides, oligonucleotides, and fragments of the invention and a nucleic acid sequence of interest will be at least 80%, and more typically with preferably increasing homologies of at least 85%, 90%, 95%, 99%, and 100%. Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program. The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The following terms are used to describe the sequence relationships between two or more polynucleotide or amino acid sequences: “reference sequence”, “comparison window”, “sequence identity”, “percentage of sequence identity”, and “substantial identity”. A “reference sequence” is a defined sequence used as a basis for a sequence comparison a reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity. A “comparison window”, as used herein, refers to a conceptual segment of at least 18 contiguous nucleotide positions or 6 amino acids wherein a polynucleotide sequence or amino acid sequence may be compared to a reference sequence of at least 18 contiguous nucleotides or 6 amino acid sequences and wherein the portion of the polynucleotide sequence in the comparison window may comprise additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman Adv. Appl. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), Geneworks, or MacVector software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology—A Synthesis (2nd Edition, E. S. Golub and D. R. Gren, Eds., Sinauer Associates, Sunderland7 Mass. (1991)). Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present invention. Examples of unconventional amino acids include: 4 hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Similarly, unless specified otherwise, the left-hand end of single-stranded polynucleotide sequences is the 5′ end the left-hand direction of double-stranded polynucleotide sequences is referred to as the 5′ direction. The direction of 5′ to 3′ addition of nascent RNA transcripts is referred to as the transcription direction sequence regions on the DNA strand having the same sequence as the RNA and which are 5′ to the 5′ end of the RNA transcript are referred to as “upstream sequences”, sequence regions on the DNA strand having the same sequence as the RNA and which are 3′ to the 3′ end of the RNA transcript are referred to as “downstream sequences”.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity.

Preferably, residue positions which are not identical differ by conservative amino acid substitutions.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine valine, glutamic-aspartic, and asparagine-glutamine.

As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the invention.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W.H. Freeman and Company, New York (1984)); Introduction to Protein Structure (C. Branden and J. Tooze, eds., Garland Publishing, New York, N.Y. (1991)); and Thornton et at. Nature 354:105 (1991).

The term “polypeptide fragment” as used herein refers to a polypeptide that has an amino terminal and/or carboxy-terminal deletion, but where the remaining amino acid sequence is identical to the corresponding positions in the naturally-occurring sequence deduced, for example, from a full length cDNA sequence. Fragments typically are at least 5, 6, 8 or 10 amino acids long, preferably at least 14 amino acids long’ more preferably at least 20 amino acids long, usually at least 50 amino acids long, and even more preferably at least 70 amino acids long. The term “analog” as used herein refers to polypeptides which are comprised of a segment of at least 25 amino acids that has substantial identity to a portion of a deduced amino acid sequence and which has specific binding to CD3, under suitable binding conditions. Typically, polypeptide analogs comprise a conservative amino acid substitution (or addition or deletion) with respect to the naturally-occurring sequence. Analogs typically are at least 20 amino acids long, preferably at least 50 amino acids long or longer, and can often be as long as a full-length naturally-occurring polypeptide.

Peptide analogs are commonly used in the pharmaceutical industry as non-peptide drugs with properties analogous to those of the template peptide. These types of non-peptide compound are termed “peptide mimetics” or “peptidomimetics”. Fauchere, J. Adv. Drug Res. 15:29 (1986), Veber and Freidinger TINS p. 392 (1985); and Evans et al. J. Med. Chem. 30:1229 (1987). Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from the group consisting of: —CH 2NH—, —CH═CH-(cis and trans), —COCH 2-, CH(OH)CH 2-, and —CH 2SO—, by methods well known in the art. Systematic substitution of one or more amino acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992)); for example, by adding internal cysteine residues capable of forming intramolecular disulfide bridges which cyclize the peptide.

The term “agent” is used herein to denote a chemical compound, a mixture of chemical compounds, a biological macromolecule, or an extract made from biological materials.

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., 3H, 14C, 15N, 35S, 90T, 99Tc, 111In, 125I, 131I), fluorescent labels (e.g., FITC, rhodamine, lanthanide phosphors), enzymatic labels (e.g., horseradish peroxidase, p-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance. The term “pharmaceutical agent or drug” as used herein refers to a chemical compound or composition capable of inducing a desired therapeutic effect when properly administered to a patient.

Other chemistry terms herein are used according to conventional usage in the art, as exemplified by The McGraw-Hill Dictionary of Chemical Terms (Parker, S., Ed., McGraw-Hill, San Francisco (1985)).

The term “antineoplastic agent” is used herein to refer to agents that have the functional property of inhibiting a development or progression of a neoplasm in a human, particularly a malignant (cancerous) lesion, such as a carcinoma, sarcoma, lymphoma, or leukemia. Inhibition of metastasis is frequently a property of antineoplastic agents.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present.

Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The amino acids encompassing the complementarity determining regions (CDR) are as defined by E. A. Kabat et al. (See Kabat, E A, et al., Sequences of Protein of immunological interest, Fifth Edition, US Department of Health and Human Services, US Government Printing Office (1991)).

### EXAMPLES

The following examples are provided for illustrative purposes only and are not to be construed as limiting upon the present invention.

### Example 1: Development of a Combination of Antibodies for Treatment of Diabetes

Type 1 diabetes (T1D) is caused by the autoimmune destruction of insulin-producing beta cells in the islets of Langerhans, which leads to dependence on exogenous insulin for survival. Despite improvements in care, the desired glycemic targets are not achieved in most patients with type 1 diabetes, and an increased risk of complications and death persists. In genetically susceptible persons, type 1 diabetes progresses through asymptomatic stages before the development of overt hyperglycemia. These stages are characterized by the appearance of autoantibodies (stage 1) and then dysglycemia (stage 2). In type 2 diabetes, metabolic responses to a glucose load are impaired and insulin treatment is not needed. Several immune interventions, when studied in patients with recent-onset clinical type 1 diabetes, have been reported to delay the decline in beta-cell function. One promising type of therapy appears to be Fc receptor-nonbinding anti-CD3 monoclonal antibodies.

The destruction of insulin producing beta cells is primarily by autoimmune disorder which can be corrected by anti-CD3 antibodies. Anti-CD3 antibodies, especially Foralumab may be used by subcutaneous administration to delay the onset and potentially cure diabetes (type 1 and 2). Particularly, this invention is directed to subcutaneous administration of Foralumab either alone or in combination with other anti-inflammatory drugs e.g., anti-IL-6 receptor mAbs to delay the onset of diabetes in juvenile patients.

### Example 2: Development of a Combination of Antibodies for Treatment of Liver Diseases

Liver pathologies (fibrosis, cirrhosis, alcoholic, non-alcoholic diseases (NAFLD and NASH) and hepatocellular carcinoma) represent one of the most common causes of death worldwide. A number of genetic and environmental factors contribute to the development of liver diseases. Interleukin-6 (IL-6) is a pleiotropic cytokine, exerting variety of effects on inflammation, liver regeneration, and defense against infections by regulating adaptive immunity. Due to its high abundance in inflammatory settings, IL-6 is often viewed as a detrimental cytokine. However, accumulating evidence supports the view that IL-6 has a beneficial impact in numerous liver pathologies, due to its roles in liver regeneration and in promoting an anti-inflammatory response in certain conditions. IL-6 promotes proliferation, angiogenesis and metabolism, and downregulates apoptosis and oxidative stress; together these functions are critical for mediating hepatoprotection. IL-6 is also an important regulator of adaptive immunity where it induces T cell differentiation and regulates autoimmunity. It can augment antiviral adaptive immune responses and mitigate exhaustion of T cells during chronic infection.

Liver diseases NAFLD and NASH may also be due to autoimmune dysfunction resulting disrupted mobilization of bile salts to liver, which causes excessive fat deposit as in case of NAFLD and NASH. This autoimmune dysfunction in bile duct may be corrected by treatment with anti-CD3 antibody. In addition, anti-CD3 antibodies may also reduce liver inflammation. Thus, combination of anti-CD3 with anti-IL-6/IL-6R mAbs are expected to ameliorate symptoms of these liver diseases. Subcutaneous administration of these mAbs is critical to reduce toxicities, which may result from combination of these antibodies administered intravenously.

### Example 3: Subcutaneous Administration with Combination of Anti-CD3 and Anti-II-6 MAbs for Treatment of Rheumatoid Arthritis

Rheumatoid arthritis (RA) is a chronic inflammatory disease associated with loss of immune tolerance. Anti-CD3 monoclonal antibodies have shown promise as a potential therapy (principally for type 1 diabetes) due to their potential to re-establish immune tolerance, partly through boosting regulatory T cells (Treg). However, its pathway into the clinical trials has been hampered due to variable clinical responses and the release of inflammatory cytokines causing flu-like symptoms. Subcutaneous administration of anti-CD3 mAbs (e.g., Foralumab) at a reduce dose in combination with other anti-inflammatory drugs may be useful. Current understanding of the immunopathogenesis of rheumatoid arthritis (RA) suggest that this inflammatory disease also has a component of autoimmune dysfunction. Thus, subcutaneous administration of anti-CD3 (e.g., Foralumab) with anti-IL-6/IL-6R mAbs could be very attractive strategy for treatment of RA. Inhibitors of IL-6 were successful in animal models of autoimmune disease paving the way for subsequent studies in humans. The greatest experience to date has been with tocilizumab, a humanized monoclonal antibody specific for the IL-6 receptor (IL-6R). Beginning with open label studies, and progressing through larger and more rigorous controlled trials, tocilizumab, an anti-IL-6R mAb, has been shown to have significant Efficacy in patients with RA. Additional studies analyzing its effects in varied populations of RA patients, as well as greater detail concerning its longer-term tolerability and safety, will help define the ultimate role of tocilizumab and other future inhibitors of IL-6 activity as potential therapies for RA. Additionally, levels of IL-6 in blood as well as in synovial fluid is known to be in excess. Therefore, anti-IL-6/IL-6R antibody that recognizes IL-6 and its soluble (sIL-6R) and membrane-bound (mIL-6R) receptors is expected to be very useful for treatment of RA. Since TZLS-501, a fully human mAb that recognizes both IL-6 receptors and IL-6, can be used for treatment with RA either alone or in combination. TZLS-501 injection directly at the site of joint inflammation may provide rapid relief by depleting IL-6 levels in synovial fluids.

### Example 4: In Vivo Pharmacokinetics of TZLS-401 (NI-0401, Foralumab)

The aim of this study was to determine the bioavailability of foralumab following SC versus IV administration. For this purpose, LCD3 transgenic mice were used to study the pharmacokinetic (Cmax and Tmax) and pharmacodynamic profiles of foralumab following a single IV or SC administrations.

### Animals and Treatment

The LCD3 transgenic mice used express human CD3ε chains on their T cells and are on a Balb/c genetic background. For evaluation of the IV effects, animals received either placebo or foralumab via retro-obital administration. Animals were sacrificed prior to obtaining blood (for plasma and leukocytes) and skin.

The doses used to evaluate the two routes of administration were 0.3 mg/kg IV (i.e., IV1×), 0.3 mg/kg SC (i.e., SC1×) and 0.6 mg/kg SC (i.e., SC2×). The choice of 0.3 mg/kg was based on previous results that 0.3 mg/kg IV induces a 70% reduction of T cells in the blood and 90% modulation of human CD3 on circulating T cells. The two dose levels tested for SC administration, i.e., the same as IV and 2-fold higher, were chosen in order to compare Cmax with IV as well as evaluate if a dose relationship with SC administration would be observed. A dose volume of 2.5 mL/kg was administered by manual injection as a bolus. Individual volume was based on individual body weight.

### Study Design

A single dose of test article was administered either subcutaneously or intravenously.

Pharamcokinetic and pharmacodynamic results obtained from animals treated IV or SC with foralumab were compared. The PD effects analyzed were: The expression levels of human CD3R at the cell surface of peripheral blood CD4+ T lymphocytes and the absolute count of CD4 and CD8 T cells in peripheral blood.

Histopathological analyses of skin samples collected at the injection site from animals treated subcutaneously with foralumab were carried out on formalin-fixed skin of the injection sites from placebo or foralumab—treated LCD3 transgenic mice.

### Test Methods

### Characterization of the Pharmacokinetic Profile of Foralumab 1 Injected Intravenously or Subcutaneously

Test article concentrations was measured in plasma samples of LCD3 transgenic mouse at various time points. Blood samples was collected in Plasma Separator Tubes (BD, PST™) and plasma was prepared by centrifugation. The plasma was then separated into aliquots of 40-50 μL and frozen (≤−80° C.). In order to measure the concentrations of foralumab in mouse plasma, a ligand-binding assay was developed using the Gyrolab technology. Briefly, biotinylated mouse anti-NI-0401 idiotype (4D10 clone) was immobilized on streptavidincoated beads (Bioaffy CD200), test sample added at a ⅕ final dilution in mouse plasma and the presence of foralumab detected using an Alexa fluor 647-conjugated mouse anti-foralumab Fcγ specific antibody (6F9 clone, anti-AE antibody).

### Characterization of the Pharmacodynamic Profile of NI-0401 Injected Intravenously or Subcutaneously

Human CD3e modulation analysis: modulation is defined as the percentage of CD3 molecules relative to baseline levels. It is expressed as the percentage of CD3 molecules removed from the T-cell surface per cell following foralumab treatment.

Briefly, whole blood from mice injected IV or SC with foralumab were collected by intracardiac puncture under terminal anesthesia. Blood samples were incubated with anti-CD4 APC and anti-Human CD3ε-PE for 30 min at 4° C. in the dark. After 2 wash, cells were resuspended in 150 μL of PBS-2% PFA before FACS analysis. Whole blood sample from placebo (PBS) IV injected mice were used as control to generate the baseline value. Practically, cells are ‘gated’ for CD4+CD45+ cells and then the expression of human CD3ε molecules expressed at the T-cell surface is measured by FACS using MFI values. The results are reported as a percentage of CD3 expression lost (human CD3ε modulation) after foralumab administration with time as compared to the placebo control (PBS) injected IV.

After intracardiac puncture under terminal anesthesia, whole blood samples were incubated in Trucount tubes containing mixture of anti-mouse CD3, CD4, CD8 and CD45 conjugated mAbs. A total volume of 50l of blood was incubated for 30 min at room temperature in the dark, in Trucount tubes containing a mixture of anti-mouse CD3-FITC, anti-mouse CD8-PE, anti-mouse CD4-PercP and anti-mouse CD45-APC conjugated-mAbs. A volume of 450 μl of “Lysis no wash solution” was added. After 30 min of incubation at room temperature in the dark, cells were then analyzed by 4-colors flow cytometry. CD45-positive cells were selectively gated to evaluate the count of CD4+ and CD8+ cells.

Histopathological Observations of Skin Samples Collected at the Injection Site from Animals Treated Subcutaneously with Foralumab

Skin samples measuring about 0.5-0.8 cm² were cut with a scalpel at the site of injection and collected from mice injected subcutaneously with the test article for histological assessment. Samples were fixed by immersion in PFA 4% for histopathological analysis Results

### Characterization of the Pharmacokinetic Profile of Foralumab Injected Intravenously or Subcutaneously.

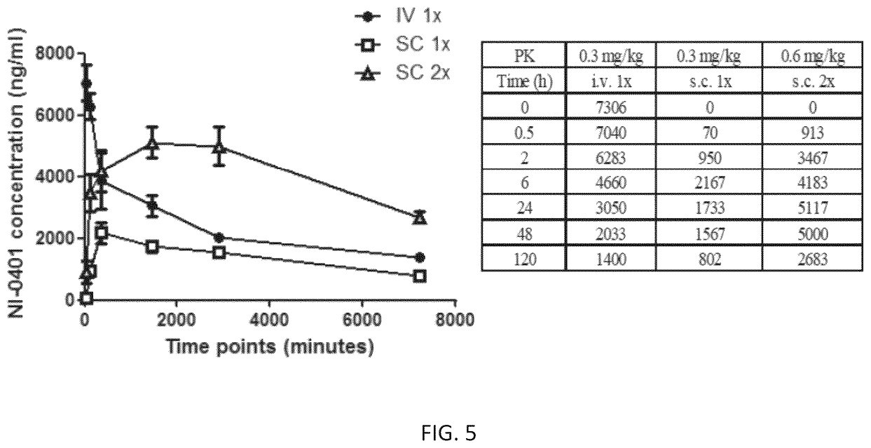

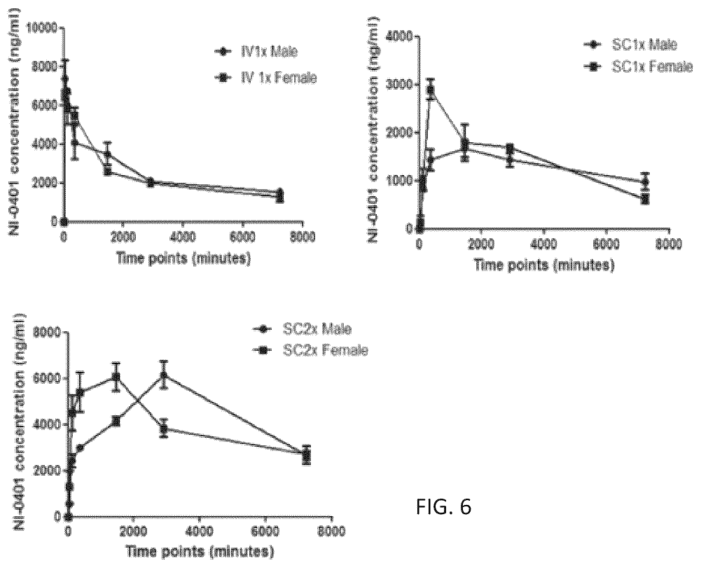

FIG. 5 and Table 5 contain the data from the analysis the PK profile of foralumab injected into LCD3 transgenic mice either IV or SC. As expected, the IV dose demonstrates a higher Cmax in blood that is not reached via SC administration with equivalent or 2 fold higher concentrations. The equivalent mg/kg dose given IV or SC (i.e., 1×; 0.3 mg/kg) shows a similar terminal phase profile. Increasing the dose 2 fold (i.e., 2×; 0.6 mg/kg) and administering SC provides a higher level of exposure and longer terminal half-life of the drug in the blood. FIG. 6 shows the comparison of the male versus female injected mice for the three groups. Overall, relatively similar profiles are observed.

TABLE 5
Pharmacokinetic parameters of foralumab injected IV or SC

| Dose | | Tmax | Cmax | AUC (0-t) | AUC (0-inf.) | λz | Half-life | | Points | Bioavail. | |
|---|---|---|---|---|---|---|---|---|---|---|---|
| (mg/kg) | Route | (h) | (ng/mL) | (ng · h/mL) | (ng · h/mL) | (/h) | (h) | No. | (%) | (h) | MRT |
| 0.3 | i.v. | 1× | 0 | 7306 | 289439 | 476318 | 0.00749 | 93 | 3 | 100 | 127 |
| 0.3 | s.c. | 1× | 6 | 2167 | 167001 | 261271 | 0.0085 | 81 | 4 | 55 | 118 |
| 0.6 | s.c. | 2× | 24 | 5117 | 500505 | 873420 | 0.0072 | 96 | 3 | 92 | 142 |

### Characterization of the Pharmacodynamic Profile of Foralumab Injected Intravenously or Subcutaneously

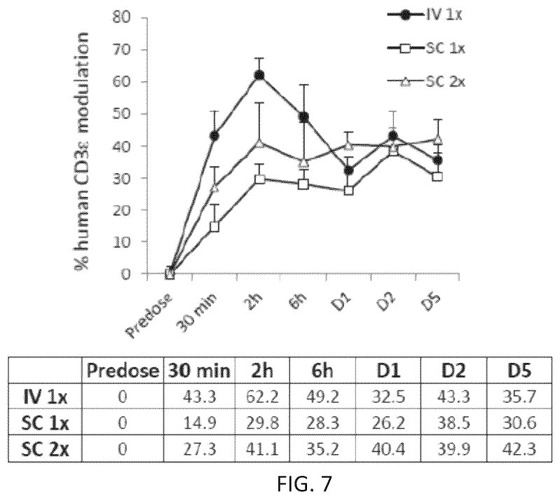

The modulation of CD3ε expression at the surface of circulating T cells occurs quickly, i.e., within 2 hours of injection for both IV and SC routes of administration (FIG. 7). The levels of human CD3ε are maximally affected by the IV route. A dose response effect is observed using the two doses administered SC with the higher dose causing more modulation.

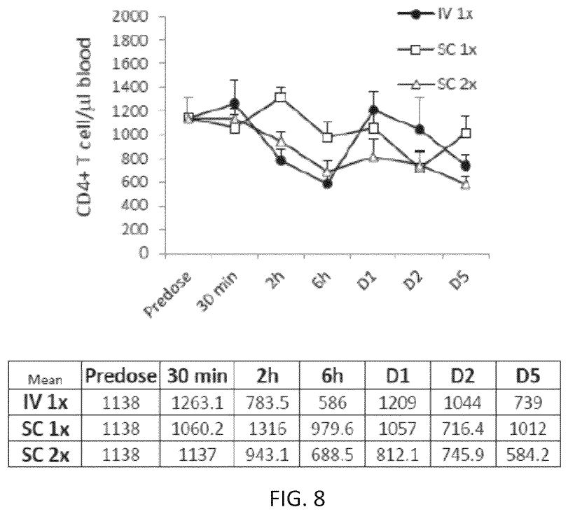

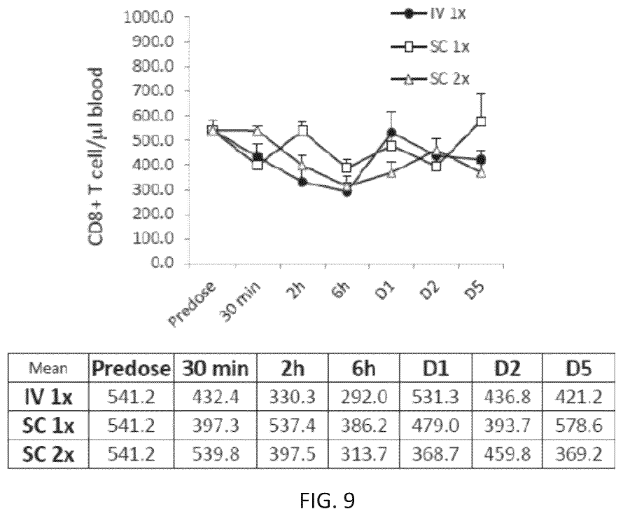

Next, the level of T cells in the blood was assessed. As expected, the loss of CD4+(FIG. 8) and CD8ε (FIG. 9) T cells from the blood was transient with the maximum effect of produced by the IV route. A dose response effect is observed using the two doses administered SC with the higher dose providing a similar T cell sequestration (i.e., loss from the blood) to the IV route.

Histopathological Observations of Skin Samples Collected at the Injection Site from Animals Treated with Placebo or Foralumab

No histopathological changes related to treatment with foralumab were observed in any of the skin samples examined. A small number of findings, distributed across the groups, were present at the injection sites, principally at 24-48 hours after treatment. These changes were mild in severity and are considered to be related to the injection procedure. It is concluded that foralumab was well tolerated at both doses and comparable to the placebo.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims. The purpose of this study was to.

We claim:

1. A method of treating or alleviating a symptom of a pulmonary disease in a subject in need thereof comprising administering to the subject a first and a second dose of an antibody that recognizes the Interleukin-6 (IL-6) and IL-6 receptor (IL-6R) complex (IL-6Rc), wherein the antibody that recognizes IL-6Rc (anti-IL-6Rc antibody) comprises a VH CDR1 region comprising the amino acid sequence of SEQ ID NO: 15, a VH CDR2 region comprising the amino acid sequence of SEQ ID NO: 32, a VH CDR3 region comprising the amino acid sequence of SEQ ID NO: 35, a VL CDR1 region comprising the amino acid sequence of SEQ ID NO: 24, a VL CDR2 region comprising the amino acid sequence of SEQ ID NO: 25, and a VL CDR3 region comprising the amino acid sequence of SEQ ID NO: 26.

2. The method of claim 1, wherein the first administration is by inhalation and the second administration is by subcutaneous injection.

3. The method of claim 1, wherein the first administration is by subcutaneous injection and the second administration is by inhalation.

4. The method of claim 1, wherein the pulmonary disease is a pulmonary inflammatory disease.

5. The method of claim 4, wherein the pulmonary inflammatory disease is acute respiratory distress syndrome idiopathic pulmonary fibrosis (IPF), or systemic pulmonary sclerosis.

6. The method of claim 1, wherein the subject has a disease or pathology associated with coronavirus infection.

7. The method of claim 1, wherein the method further comprises subcutaneously administering to the subject a first dose of a composition comprising an anti-CD3 antibody, wherein the anti-CD3 antibody comprises a heavy chain complementarity determining region 1 (CDRH1) comprising the amino acid sequence GYGMH (SEQ ID NO: 42), a heavy chain complementarity determining region 2 (CDRH2) comprising the amino acid sequence VIWYDGSKKYYVDSVKG (SEQ ID NO: 43), a heavy chain complementarity determining region 3 (CDRH3) comprising the amino acid sequence QMGYWHFDL (SEQ ID NO: 44), a light chain complementarity determining region 1 (CDRL1) comprising the amino acid sequence RASQSVSSYLA (SEQ ID NO: 45), a light chain complementarity determining region 2 (CDRL2) comprising the amino acid sequence DASNRAT (SEQ ID NO: 46), and a light chain complementarity determining region 3 (CDRL3) comprising the amino acid sequence QQRSNWPPLT (SEQ ID NO: 47).

8. The method of claim 7, wherein the anti-CD3 antibody is a monoclonal antibody.

9. The method of claim 7, wherein the anti-CD3 antibody is fully human or humanized.

10. The method of claim 7, wherein
(a) the anti-CD3 antibody comprises a variable heavy chain amino acid sequence comprising the amino acid sequence of SEQ ID NO: 48 and a variable light chain amino acid sequence comprising the amino acid sequence of SEQ ID NO: 49 or
(b) the anti-CD3 antibody comprises a heavy chain amino acid sequence comprising the amino acid sequence of SEQ ID NO: 50 and a light chain amino acid sequence comprising the amino acid sequence of SEQ ID NO: 51.

11. The method of claim 1, wherein the antibody that recognizes IL-6Rc is a monoclonal antibody.

12. The method of claim 1, wherein the antibody that recognizes IL-6Rc is fully human.

13. The method of claim 7, wherein the anti-CD3 antibody is administered at a daily dose of 1 mg to 5 mg.

14. The method of claim 13, wherein the daily dose is administered for at least 5 consecutive days.

15. The method of claim 1, wherein the anti-IL-6Rc is administered at a daily dose of 50 mg to 300 mg.

16. The method of claim 15, wherein the daily dose is administered once daily.

17. The method of claim 15, wherein the daily dose is administered once every 14 days.

18. The method of claim 1, wherein the anti-IL-6Rc antibody is administered subcutaneously.

19. The method of claim 7, further comprising administering to the subject a second dose of the anti-CD3 antibody, wherein the second administration is intranasal.

20. The method of claim 7, further comprising a second administration of the anti-CD3 antibody, wherein the second administration is subcutaneous and to a different site of the body than the first administration.
